(19) United States
(12) Patent Application Publication
(10) Pub. No.: US 2012/0136209 A1
(43) Pub. Date: May 31, 2012

(54) METHODS AND DEVICES FOR PROVIDING INFORMATION USEFUL IN THE DIAGNOSIS OF ABNORMALITIES OF THE GASTROINTESTINAL TRACT

(75) Inventors: Genady Kostenich, Ramat Gan (IL); Arie Orenstein, Tel Aviv (IL)

(73) Assignee: TEL HASHOMER MEDICAL RESEARCH INFRASTRUCTURE AND SERVICES, LTD., Ramat Gan (IL)

(21) Appl. No.: 13/388,371

(22) PCT Filed: Aug. 4, 2010
(86) PCT No.: PCT/IB2010/053539

§ 371 Date: Feb. 15, 2012

Related U.S. Application Data
(60) Provisional application No. 61/231,350, filed on Aug. 5, 2009.

(51) Int. Cl. A61B 1/04

(52) U.S. Cl. 600/109

(57) ABSTRACT

Disclosed are methods useful for providing information useful in the diagnosis of gastrointestinal abnormalities as well as ingestible devices useful for providing information useful in the diagnosis of gastrointestinal abnormalities.

### RELATED APPLICATION

The present application gains priority from U.S. Provisional Patent Application No. 61/231,350 filed 5 Aug. 2009, which is included by reference as if fully set forth herein.

### FIELD AND BACKGROUND OF THE INVENTION

The invention, in some embodiments, relates to the field of medical diagnosis of the gastrointestinal tract, and more particularly but not exclusively, to methods and devices for providing information useful in the diagnosis of abnormalities of the gastrointestinal tract (gastrointestinal abnormalities).

Ingestible imaging devices for inspecting the gastrointestinal tract are known, for example the Pillcam™ available from Given Imaging (Yokneam, Israel). Such capsule-shaped devices generally include an illumination source and an imaging component (a digital camera) at one or both (distal and proximal) ends. As known in the art of digital photography, the image-acquisition portion of such a camera is a planar array of light sensitive sensors (e.g., CCD, CMOS). For use, such an imaging device is swallowed by a subject and the device is propelled through the gastrointestinal tract by peristalsis. While passing through the gastrointestinal tract, the camera acquires images of the gastrointestinal tract and wirelessly transmits the images to a recording device. The images of the gastrointestinal tract are subsequently inspected by a health care professional, as a lengthy video, for evidence of gastrointestinal abnormalities such as bleeding, polyps, cancers and lesions.

Such ingestible imaging devices have disadvantages. Ingestible imaging devices are expensive, requiring complex and expensive cameras and often requiring small moving parts such as lenses in order to acquire diagnostically-useful images. Ingestible imaging devices have high power requirements for operating the camera and an illumination source bright enough to allow acquisition of the images. The camera of such imaging devices is necessarily directed in parallel to the lumen but has limited field of view of the intestinal wall where gastrointestinal abnormalities are located. Ingestible imaging devices produce large amounts of image (video) data. As a result, data acquisition and transmission is not trivial, must be performed continuously and requires a significant amount of power. The large amount of data cannot be reviewed automatically and instead requires a time-consuming review by a skilled health-care professional, a factor that raises the cost of using such devices. Even the most highly skilled health-care professional is only able to identify relatively large abnormalities that are visible under the poor intraluminal lighting conditions so that small abnormalities and abnormalities that have certain colors often remain undetected.

It would be useful to have methods and ingestible devices that provide information useful for the diagnosis of gastrointestinal abnormalities that are devoid of at least some of the disadvantages of known ingestible imaging devices.

### SUMMARY OF THE INVENTION

Some embodiments of the invention relate to methods and devices for providing information useful for the diagnosis of gastrointestinal abnormalities by determining the intensity of at least one specified wavelength of light reflected, in some embodiments diffusely reflected light, from a portion of an intestinal wall, that in some embodiments are devoid of at least some of the disadvantages of known ingestible imaging devices.

In some embodiments, the relative intensity of at least two specified wavelengths of light (diffusely) reflected from a portion of the intestinal wall is determined and provides information useful for the diagnosis of gastrointestinal abnormalities.

Thus, according to an aspect of some embodiments of the invention, there is provided a method for providing information useful for the diagnosis of gastrointestinal abnormalities, comprising:

a) illuminating an area of an in vivo gastrointestinal tract of a living mammal with light, wherein the illuminating is outwards from inside the gastrointestinal tract lumen;

b) without acquiring an image of the area, determining an intensity of at least one specified wavelength of light after the light is reflected from the area of the gastrointestinal tract; and

c) providing information related to the intensity of light indicative of a potential gastrointestinal abnormality in the area. In some embodiments, the intensity determined is of diffusely reflected light.

In some embodiments, the information provided is a comparison of at least two respective intensities of at least two different specified wavelengths reflected from the same area.

In some embodiments, the information provided is the intensity of the specified wavelength of light compared to an intensity of light of the same specified wavelength after the light has been reflected from a different area of the gastrointestinal tract.

According to an aspect of some embodiments of the invention, there is also provided an ingestible device useful for providing information useful for the diagnosis of gastrointestinal abnormalities, comprising:

a) an ingestible casing having a device axis, a body section, a distal end and a proximal end;

b) inside the casing, an illuminator configured to project light radially outwards through an illuminator window of the housing; and

c) inside the casing, at least one light-detection assembly configured to determine the intensity of at least one specified wavelength of light passing through an associated detector window (without acquiring an image).

According to an aspect of some embodiments of the invention, there is also provided an illuminator useful for projecting light in a radially-outwards direction in a 360° circumferential section comprising:

a) a light source for projecting light; and

b) a radial diffuser having a diffuser axis, a first face, a second face and a substantially circular circumferential outer edge coaxial with the central diffuser axis,
wherein the light source is configured to project light into the radial diffuser; and
wherein at least a portion of the light projected into the radial diffuser from the light source radiates radially-outwards through the circumferential edge.

According to an aspect of some embodiments of the invention, there is also provided a diffraction grating, comprising:

a substantially conical-section surface having an axis; and

on the surface, periodic features

wherein the surface and the features are configured to reflect light impinging on the surface substantially perpendicularly to the axis at a wavelength-dependent angle in the general direction of the axis so that the diffraction grating functions as an optically dispersive element.

Unless otherwise defined, all technical and scientific terms used herein have the same meaning as commonly understood by one of ordinary skill in the art to which the invention pertains. In case of conflict, the specification, including definitions, will control.

As used herein, the terms “comprising”, “including”, “having” and grammatical variants thereof are to be taken as specifying the stated features, integers, steps or components but do not preclude the addition of one or more additional features, integers, steps, components or groups thereof These terms encompass the terms “consisting of” and “consisting essentially of”.

As used herein, the indefinite articles “a” and “an” mean “at least one” or “one or more” unless the context clearly dictates otherwise.

### BRIEF DESCRIPTION OF THE FIGURES

Some embodiments of the invention are herein described with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIGS. 1A-1C depict results of experimentally-acquired spectra of normal and abnormal gastrointestinal tissue, demonstrating aspects of the teachings herein;

FIGS. 2A-2C are schematic depictions of an embodiment of a device as described herein configured for determining the intensity of only one specified wavelength of light;

FIGS. 2D and 2E depict typical information acquired using a device depicted in FIG. 2A;

FIG. 3 is a schematic depiction of the device of FIG. 2 in use inside a gastrointestinal tract;

FIGS. 4A-4C are schematic depictions of embodiments of devices as described herein configured for determining the intensity of three specified wavelength of light with three light-detection assemblies;

FIGS. 4D and 4E depict typical information acquired using a device depicted in FIG. 4A;

FIG. 5 is a schematic depiction of an embodiment of a device as described herein configured for determining the intensity of three specified wavelength of light with six light-detection assemblies;

FIG. 6 is a schematic depiction of an embodiment of a device as described herein configured for determining the intensity of three specified wavelength of light with a single light-detection assembly;

FIG. 7 is a schematic depiction of an embodiment of a device as described herein configured for determining the intensity of a plurality of specified wavelengths of light with a single light-detection assembly;

FIGS. 8A-8E are schematic depictions of an embodiment of a device as described herein used in implementing an embodiment of a method as described herein inside a gastrointestinal tract; and

FIG. 9 is a schematic depictions of an embodiment of device as described herein configured for determining the intensity of three specified wavelength of light with three light-detection assemblies in accordance with the teachings herein, and further including two imaging cameras.

### DESCRIPTION OF SOME EMBODIMENTS OF THE INVENTION

Some embodiments of the invention relate to methods and devices for providing information useful for the diagnosis of gastrointestinal abnormalities by determining the intensity of at least one specified wavelength of light (diffusely) reflected from an area of the gastrointestinal tract without acquiring an image of the area. The area of a gastrointestinal tract is, depending on the embodiment, any portion of a gastrointestinal tract that is downstream of the pylorus, e.g, the duodenum, small intestine or large intestine.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

Known ingestible imaging devices for inspection of the gastrointestinal tract have a number of disadvantages as discussed in the introduction hereinabove.

Herein are disclosed methods and devices for providing information useful for the diagnosis of gastrointestinal abnormalities by determining the intensity of at least one specified wavelength of light (diffusely) reflected from an area of the gastrointestinal tract (e.g., intestinal wall).

To understand some embodiments of the method, one must consider the process of reflection, both diffuse reflection and specular reflection.

Some light that illuminates an area of intestinal wall penetrates into the tissue and is absorbed by chromophores in the tissue. As any given chromophore absorbs different wavelengths of light with different efficiencies and as the presence, nature and concentration of chromophores is tissue-type dependent, different tissue types absorb different wavelengths of light differently.

Light that penetrates into the tissue is also scattered inside the tissue, reflected and refracted in many directions from various features such as cells, organelles and interstitial tissue where some of the light is ultimately diffusely reflected out of the tissue back in the general direction of the illumination. The degree of scattering and diffuse reflection is dependent on the concentration, size and shape of the reflecting features as well as the refractive index of the various tissue components. Due to the ranges of refractive indices, concentrations as well as size of scattering features in gastrointestinal tissue, the degree of diffuse reflection of visible light and near-infrared light (400-800 nm) is wavelength-dependent.

It has been found and is herein disclosed that the differences of light absorption, scattering and diffuse reflection in normal compared to some abnormal gastrointestinal tissue is sufficient to provide information that can be useful for diagnosis of gastrointestinal abnormalities.

Further, for a given light-detector, the detected intensity of both specular and diffuse reflection from a tissue surface is dependent on the distance of the detector from the surface: the further a surface, the lower the intensity of reflected light independent of wavelength.

According to an aspect of some embodiments of the invention, there is provided a method for providing information useful for the diagnosis of gastrointestinal abnormalities, comprising:
a) illuminating an area of an in vivo gastrointestinal tract of a living mammal (a human or non-human mammal) with light, wherein the illuminating is outwards from inside the gastrointestinal tract lumen;
b) without acquiring an image of the area, determining an intensity of at least one specified wavelength of light after the light is (diffusely) reflected from the area of the gastrointestinal tract; and
c) providing information related to the intensity of light indicative of a potential gastrointestinal abnormality in the area, for example to a person such as a health-care professional.

In some embodiments, the mammal is unsedated, preferably ambulatory, whether in a clinical or (more preferably) non-clinical setting.

In some embodiments, the intensity determined is of diffusely reflected light. In some embodiments, the intensity determined is substantially exclusively of diffusely reflected light.

The provided information may then be used, alone or together with other information regarding the living mammal, to diagnose a gastrointestinal abnormality, for example bleeding, cancers, invasive adenocarcinoma, adenomas, adenomatous polyps, benign polyps and hyperplastic polyps but also other abnormalities.

In some embodiments, the provided information relates to wavelength-dependent abnormalities, that is to say, have characteristics to absorb and scatter different wavelengths of light differently. In some embodiments, the abnormalities detected are distance (from an illuminator and to a light-detection assembly) sensitive, that is to say the intensity of the reflected light changes with distance, for example polyps and other abnormalities having an abnormal shape, usually protruding into the intestinal lumen.

Thus, in some embodiments, identification of tissue as potentially abnormal is by wavelength-dependent reflection characteristics and does not require acquisition and analysis of an image as known in the art. Accordingly, some embodiments of the method described herein may be considered a form of spectroscopy. Useful background art for greater understanding some aspects of some embodiments of the method may be found, for example, in Dhar M et al, Gastrointestinal Endoscopy 2006, 63(2), 257.

In some embodiments, when an area of gastrointestinal tissue is illuminated with light some of the light penetrates into the tissue and undergoes both absorption and scattering. The intensity of a specified wavelength of light that is ultimately diffusely reflected and detected is tissue-type dependent. By determining an intensity of a specified wavelength of light diffusely reflected from an area of tissue and comparing the determined intensity to some reference value (e.g., an absolute value, an intensity of light having the same wavelength reflected from a different area, an intensity of light having a different wavelength reflected from the same area) it is possible to identify the area of tissue as potentially abnormal.

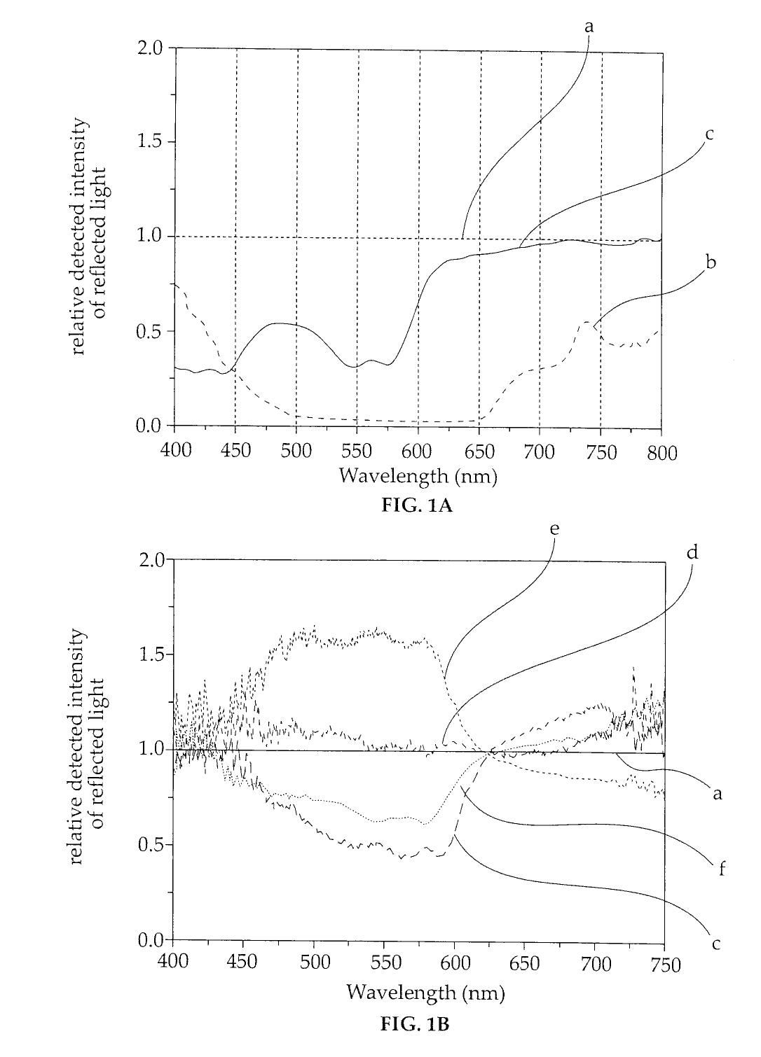

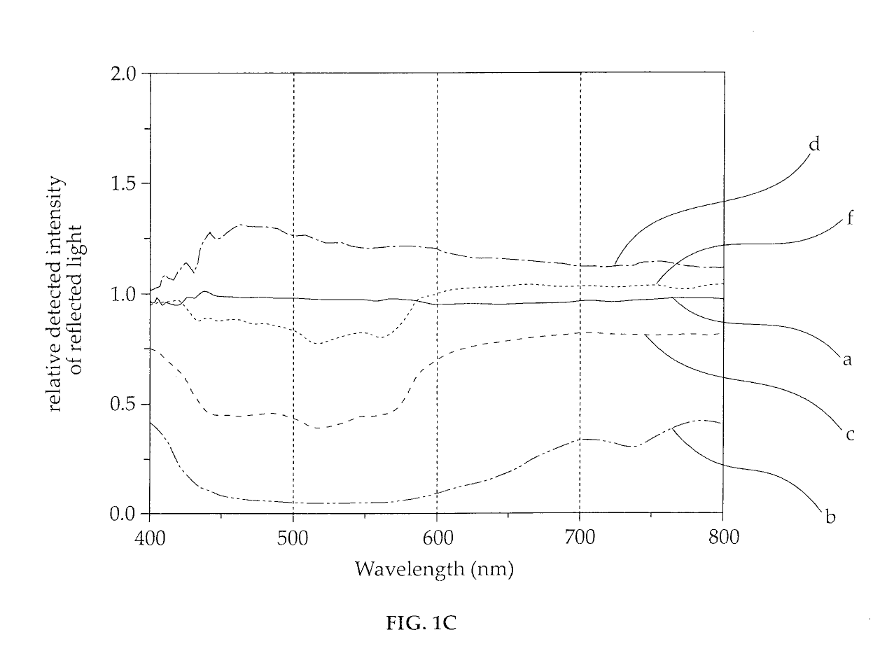

In FIGS. 1A-1C results are shown of actual experiments performed (as detailed in the Example section) on normal and abnormal gastrointestinal tissue demonstrating the wavelength and tissue-type dependence of diffuse reflection.

In a first experiment, the wavelength dependence of diffuse reflection from intestinal abnormalities relative to normal intestinal mucosa was examined in a manner simulating the use of an ingestible device as described herein on excised intestinal tissue. The results of the experiment are shown in FIG. 1A, where the relative detected intensities of light diffusely reflected from normal mucosa (plot ‘a’), blood (plot ‘b’) and invasive adenocarcinoma (plot ‘c’) normalized relative to light reflected from normal mucosa at wavelengths between 400 nm and 800 nm are shown.

In a second experiment, the wavelength dependence of diffuse reflection from various intestinal abnormalities related to invasive adenocarcinoma relative to normal intestinal mucosa was examined in a manner simulating the use of an ingestible device as described herein in vivo. The results of the experiment are shown in FIG. 1B, where the relative detected intensities of light diffusely reflected from normal mucosa (plot ‘a’), invasive adenocarcinoma (plot ‘c’), a hyperplastic polyp (plot ‘d’), an adenomatous polyp (plot ‘e’) and adenoma (plot ‘f’) normalized relative to light diffusely reflected from normal mucosa at wavelengths between 400 nm and 750 nm are shown.

In a third experiment, the wavelength dependence of diffuse reflection from various intestinal abnormalities relative to normal intestinal mucosa was examined in a manner simulating the use of an ingestible device as described herein on excised intestinal tissue. The results of the experiment are shown in FIG. 1 C, where the relative detected intensities of light diffusely reflected from normal mucosa (plot ‘a’), blood (plot ‘b’), invasive adenocarcinoma (plot ‘c’), a hyperplastic polyp (plot ‘d’) and adenoma (plot ‘f’) normalized relative to light diffusely reflected from normal mucosa at wavelengths between 400 nm and 800 nm are shown.

As apparent from FIGS. 1A-1C, different tissue types diffusely reflect light in a characteristic, identifiable and wavelength-dependent fashion so that information relating to the intensity of at least one specified wavelength of diffusely reflected light provided in accordance with the teachings herein may be useful in assisting diagnosis of some types of gastrointestinal abnormalities.

In some embodiments, at least one of the specified wavelengths is a wavelength having a high tissue-type dependent diffuse reflection for a gastrointestinal abnormality and can be considered a diagnostic wavelength for that gastrointestinal abnormality. In some embodiments, at least one of the specified wavelengths is a wavelength having a low tissue-type dependent diffuse reflection for a gastrointestinal abnormality. “Low” and “high” are qualitative terms that are easily understood by a person having ordinarily skill in the art, for example, by consulting FIGS. 1A-1C or through minor, not undue, experimentation.

For example, from FIGS. 1A-1C, it is seen that various wavelengths between 450 and 700 nm can be diagnostic wavelengths for bleeding, invasive adenocarcinomas, adenomas, and adenomatous polyps when compared to normal tissue.

In some embodiments, the intensity of at least two different specified wavelengths of light reflected from the same area is determined. The relative intensities of the reflections of the two different wavelengths are compared and, as apparent from FIGS. 1A-1C, may be indicative of abnormal tissue.

For example, a strong reduction of intensity for wavelengths between about 430 nm and about 600 nm (relative to normal tissue) when accompanied by a more moderate reduction, unchanged or increase of intensity for wavelengths between about 700 nm and about 800 nm (relative to normal tissue) may be indicative of an abnormality such as invasive adenocarcinoma but when accompanied by a strong reduction of intensity for wavelengths between about 700 nm and about 800 nm (relative to normal tissue) may be indicative of an abnormality such as bleeding.

In some such embodiments, one of the at least two specified wavelengths is diagnostic for one or more abnormalities, and another of the at least two specified wavelengths is diagnostic for at least one different abnormality.

In some such embodiments, one of the at least two specified wavelengths is diagnostic for an abnormality, and another of the at least two specified wavelengths is diagnostic for the same abnormality, providing increased confidence in a potential diagnosis.

In some such embodiments, the determination of the intensity of light of at least two specified wavelength allows comparison of the intensities of the two wavelengths to provide information useful for diagnosis.

In some such embodiments, at least one of the at least two specified wavelengths is a diagnostic wavelength (a wavelength which intensity changes are indicative of an abnormality, e.g. 550 nm indicative of invasive adenocarcinoma) and at least one of the at least two specified wavelengths is a reference wavelength (e.g., 700 nm). Determination of the intensity of a reference wavelength allows, in some embodiments, normalization of the determined intensities of the one or more diagnostic wavelengths and, in some embodiments, allows separation of diagnostically useful measurements from not-useful measurements.

In some such embodiments, the variations in intensities are wavelength independent and indicative of the lack of contact with the intestinal wall. In some such embodiments, the intensities of the diagnostic wavelength determined during contact with the intestinal wall are separated from the intensities of the diagnostic wavelength determined with no contact with the intestinal wall with reference to the determined intensities of a reference wavelength. For example, in some embodiments, the intestinal walls physically contact a measuring device only in the contracted portion of the peristaltic cycle. A similar magnitude of reduction in intensity of reflection of both 650 nm (reference) and 550 nm (diagnostic) wavelengths is indicative of no contact with the intestinal wall during that measurement. In contrast, a reduction in intensity of reflection of 550 nm while the intensity of reflection of 650 nm remains substantially constant is indicative of contact with the intestinal wall during that measurement with possible detection of a potential invasive adenocarcinoma.

In some embodiments, the information provided is the determined intensities. In some embodiments, the information provided is information calculated from the determined intensities, for example relative intensities, ratios and the like.

In some embodiments, the information provided is the intensity of a specified wavelength of light reflected from an area of the gastrointestinal tract compared to the intensity of light having the same specified wavelength reflected from a different area of the gastrointestinal tract. In some such embodiments, especially when the intensity of reflected light is determined from a plurality of discrete areas substantially simultaneously, differences in the tissue type in the a different areas or difference in the distance to the different areas can be identified. For example, as seen from FIG. 1A, when comparing the intensity of reflection from different areas, a reduced intensity of light at 550 nm may be indicative of an abnormality such as bleeding or invasive adenocarcinoma.

In some embodiments, the information provided is a comparison of at least two (in some embodiments, exactly two or exactly three) respective intensities of at least two different specified wavelengths reflected from the same area, for example the ratio of intensities of two different wavelengths is compared, where a low ratio is indicative of normal tissue and a high ratio is indicative of abnormal tissue. In some such embodiments, the comparison reduces the influence of non-wavelength dependent variations in intensity and emphasizes the influence of wavelength-dependent variations, and is therefore useful, in some embodiments, for differentiating between different tissue types. Such wavelength intensity ratios that are significantly differ (lower or higher) than of normal tissue may be indicative of an abnormality.

For example, in some embodiments the intensity of light having a wavelength of 550 nm (I550) reflected from an area is compared to that of light having a wavelength of 625 nm (I625) reflected from the same area. Intensities that are similar (I550=I625) but both lower than of normal tissue are indicative of bleeding, a significantly higher intensity of I625 compared to I550 (I625>I550) is indicative of invasive adenocarcinoma or adenoma, while a significantly higher intensity of I550 compared to I625 (I550>I625) is indicative of adenomatous polyp of hyperplastic polyp.

In some embodiments, for example when greater detection confidence is desired (both with regards to selectivity, specificity, abnormality type) or when it desired to seek and potentially detect more than one type of abnormality, the relative intensities of more than two wavelengths of reflected light are determined and compared (e.g., three, four, five and even more wavelengths). In some embodiments, the relative intensities of only the wavelengths to be compared are acquired. In some embodiments, the relative intensities of more than only the wavelengths to be compared are acquired. In some embodiments, the relative intensities of a plurality of wavelengths are acquired, in some embodiments constituting a spectrum.

In some embodiments, the method described herein is not applied to only detecting an abnormality at one specific selected area of a gastrointestinal tract.

In some embodiments of the method described herein includes scanning the surface of the gastrointestinal tract and determining the intensity of at least one specified wavelength of light reflected from each one of a plurality of areas in succession.

In some embodiments of the method described herein includes simultaneously determining the intensity of at least one specified wavelength of light reflected from each one of a plurality of areas.

In some embodiments of the method described herein includes substantially simultaneously illuminating a plurality of discrete areas of the gastrointestinal tract. In some embodiments, the plurality of discrete areas constitute a circumferential section of the gastrointestinal tract, in some embodiments a 360° circumferential section (a ring) of the gastrointestinal tract. In some embodiments, determining the intensity of at least one specified wavelength of reflected light is substantial simultaneous for the plurality of discrete areas. In some embodiments, the method comprises sequentially detecting light reflected from succeeding rings of tissue making up the gastrointestinal tract.

In some embodiments, the determined intensities are analyzed and reacted upon, for example a specific action is undertaken when potentially abnormal tissue is detected, e.g., an alarm is sounded, a marker or active pharmaceutical ingredient is administered.

In some embodiments, the provided information (e.g., determined intensities, relative intensities, ratios) is recorded. In some embodiments, the provided information is transmitted.

In some embodiments, the provided information is transmitted continuously. In some embodiments, the provided information is transmitted intermittently (e.g., less frequently than about every minute, less frequently than about every hour), for example in order to save power. In some embodiments, a portable relay device (e.g., a cellular telephone, a personal digital assistant, a dedicated transceiver) is in proximity to the of the living mammal and used as a relay, continuously or intermittently receiving the provided information and subsequently retransmitting the provided information, for example to a central location such as hospital or physician. In some embodiments, the provided information is converted into an image, for example using pipe-simulation mathematics. In some embodiments, a portable recording device (e.g., a cellular telephone, a personal digital assistant, a dedicated recorder receiver) is in proximity to the of the living mammal and used to, continuously or intermittently receive the provided information and subsequently record the information, for example on a removable storage medium such as a memory card.

The method described herein may be implemented using any suitable device, for example, in some embodiments, a device as described herein is used to implement the method.

According to an aspect of some embodiments of the invention, there is provided an ingestible device for providing information useful for the diagnosis of gastrointestinal abnormalities, the device comprising:
a) an ingestible casing having a device axis, a body section, a distal end and a proximal end;
b) inside the casing, an illuminator configured to project light radially outwards through an illuminator window of the housing, allowing illumination a region of a gastrointestinal tract wall (e.g., intestinal wall) in which the device is passing and contacting;
c) inside the casing, at least one light-detection assembly configured to determine the intensity of at least one specified wavelength of light passing through an associated detector window without acquiring an image.

For use in accordance with some embodiments of the method discussed hereinabove, the device is ingested. After passing the pylorus, the illuminator projects light radially through the associated illuminator window that in some embodiments acts as a light guide to guide light from the illuminator out of the casing, illuminating an area of the gastrointestinal wall. The projected light is reflected back through a detector window that in some embodiments acts as a light guide to guide reflected light towards the light-detection assembly where the intensity of the reflected light is determined. As discussed above with reference to the method described herein, the intensity of at least one specified wavelength of light reflected from an area in the illuminated region may be indicative of an abnormality in the area.

In the art, ingestible gastrointestinal imaging devices include an objective, usually an adjustable objective, comprising one or more lenses and/or mirrors, that form an image of an object on a two-dimensional detector array located at the focal plane of the objective. The device described herein is used for inspection of the gastrointestinal tract without acquiring an image. In some embodiments, the inspection of the gastrointestinal tract is performed without a lens. In some embodiments, the device comprises a fixed lens to concentrate light at a light detector, but not to form an image.

It is important to note that in order to acquire an image, prior art ingestible imaging devices necessarily include a camera. The camera acquires an image of a relatively large portion of the intestinal tract, generally in parallel to the intestinal lumen. As known in the art of digital cameras, each pixel of the camera detector simultaneously acquires multiple wavelengths of light from a given area (each pixel corresponding to an area, where the areas together make up the portion of the intestinal tract of which image is being acquired). Such imaging devices need relatively long acquisition time for each image frame.

In contrast, in some embodiments of the device herein, the intensity of each specified wavelength from a given area is determined in a different physical location, e.g., a different light-detection assembly, a different region of a detector of the same light-detection assembly. Thus, in some embodiment, the device is configured to determine the intensity of at least two specified wavelengths of light each at a substantially different location. In some embodiments, the device is configured to determine the intensity of at least two specified wavelengths of light, each with a substantially different detector. In some embodiments of the device herein, a light-detection assembly includes a simple, one-dimensional array of light sensors. Such detectors determine the intensity of each specified wavelength from a relatively small area and need a relatively short acquisition time at one physical location of the device.

Some embodiments of the devices described herein are relatively cheap, requiring simpler and cheaper components than known ingestible imaging devices.

Some embodiments of the devices described herein are relatively mechanically reliable compared to known ingestible imaging devices, requiring no moving parts.

Some embodiments of the devices described herein have a low power consumption compared to known ingestible imaging devices (depending on the embodiment, due to, for example, less intense illumination requirements, less acquired data, no moving mechanical parts, fewer pixels per frame), requiring smaller, cheaper and less toxic power storage units, e.g., batteries.

Like prior art ingestible imaging devices, the casing of a device is generally configured for passage, once swallowed by a mammalian subject and passing the pylorus, for transport through the gastrointestinal tract for eventual expulsion through the anus where either the distal or proximal end faces forwards, where the device axis is substantially parallel to the gastrointestinal tract lumen and where the gastrointestinal walls physically contacts the body section to propel the device by peristalsis.

In some embodiments, the body section includes a substantially parallel-walled cylindrical portion where the walls of the body section are parallel to the body axis.

In some embodiments, the distal end and/or the proximal end are streamlined for passage of the device through the gastrointestinal tract.

In some embodiments, the device comprises inside the casing a power supply for providing substantially all power for operation of the device once ingested until expelled. In some embodiments, when compared to a power supply required for known ingestible imaging devices, the power supply is relatively modest as the device has relatively lower power requirement as there is no need for imaging, no moving parts, less intense illumination, and per unit time, less data to transmit and/or record.

In some embodiments, the device comprises inside the casing, a processor, for example configured to compare the determined intensities of specified wavelengths of light.

In some embodiments, the device comprises inside the casing, a wireless transmitter for transmitting information related to the determined intensities of the specified wavelengths, such as the determined intensity of the specified wavelengths or results of comparison of the determined intensities of the specified wavelengths. In some embodiments, the wireless transmitter is, like in known ingestible imaging devices, configured for continuous transmission of the information. In some embodiments, the wireless transmitter is configured for non-continuous (e.g., intermittent or periodic) transmission of the information, in order to reduce interference potentially caused by transmission, to reduce exposure of a subject ingesting the device to radiation, and to reduce power use by the device. In some embodiments, non-continuous transmission is possible because the device does not acquire images, but only modest amounts of information. In some embodiments, the information is transmitted only to a short range (e.g., to a device carried by the subject such as a dedicated device, a cellular telephone, a personal digital assistant) and stored and/or retransmitted.

In some embodiments, the device comprises inside the casing, a memory for recording information related to the determined intensities of the specified wavelength, for example, the device includes a solid-state memory component or a removable solid-state memory component such as a micro-SD card. In some embodiments, the recorded information is substantially all the information useful for the diagnosis of abnormalities in the gastrointestinal tract determined during passage through a gastrointestinal tract. In some embodiments, recording of substantially all the useful information is possible because the device does not acquire images, but only relatively modest amounts of information.

An illuminator of a device described herein is configured to project light including one or more specified wavelengths, typically between about 400 nm and 800 nm. In some embodiments, an illuminator is configured to project monochromatic light. In some embodiments, an illuminator is configured to project polychromatic or white light that includes the specified wavelengths of light.

In some embodiments, the illuminator comprises a light source for producing light having the specified wavelengths. Any suitable light source may be used, for example a light-emitting diode. In some embodiments, such a light source is configured for producing monochromatic light. In some embodiments, such a light source is configured for producing polychromatic light. In some embodiments, such a light source is configured for producing white light. In some embodiments, the illuminator comprises a light source for producing light having wavelengths between 400 and 800 nm.

In some embodiments, a light source comprises a radial diffuser functionally associated with the light source for radially distributing light produced by the light source.

The illuminator and the illuminator window are configured together to project light in any suitable direction. In some embodiments, the illuminator and illuminator window are configured together to project light substantially perpendicularly to the device axis. In some embodiments, the illuminator and illuminator window are configured together to project light at an angle different than 90° to the device axis, e.g., in a direction towards or away from a detector window, in some embodiments not more than 10° from perpendicular to the device axis and in some embodiments not more than 5° from perpendicular to the device axis.

The illuminator is configured to project light radially allowing illumination of any shaped region of a gastrointestinal lumen. In some embodiments, the illuminator is configured to project light in circumferential section of at least about 90°, at least about 120°, and even at least about 180° at one time around the device axis, allowing substantially simultaneous illumination of an equivalent circumferential section of gastrointestinal lumen. That said, in preferred embodiments, the illuminator is configured to project light in a circumferential section that is substantially the entire 360° around the device axis, allowing substantially simultaneous illumination of substantially a 360° circumferential section (a ring) of gastrointestinal tissue.

The illuminator window through which the illuminator projects light is substantially transparent to at least the specified wavelengths. An illuminator window is of any suitable shape and construction, and is generally fashioned of glass or plastic material as known in the art of ingestible imaging devices. In some embodiments, an illuminator window is made up of two or more discrete parts assembled so that the illuminator window is continuous. In some embodiments, an illuminator window is made up of two or more discrete parts assembled where at least two of the parts are separated by a non-transparent component. That said, it is generally preferred (for reasons of ease of construction as well as to reduce the chance of leakage of gastrointestinal fluids into the casing) that the illuminator window comprises substantially a single discrete component. In some embodiments, a radial diffuser constitutes the illuminator window.

In some embodiments, the illuminator and the illuminator window together are configured that the projected light is polarized, for example, the illuminator window is a light polarization component or the illuminator comprises a light polarization component.

In some embodiments where the illuminator is configured to project light in a certain circumferential section (e.g., 120°), the illuminator window comprises an arc or disk section of at least the same circumferential section of a material substantially transparent to light having the specified wavelengths. In some embodiments, especially where the illuminator is configured to project light in a substantially 360° circumferential section, the illuminator window is a ring or a disk of a material substantially transparent to light having the specified wavelengths, in some embodiments positioned coaxial with the device axis.

A light-detection assembly of a device described herein is configured to determine the intensity of at least one specified wavelength of light passing through an associated detector window. As discussed above, specified wavelengths are typically between about 400 nm and 800 nm.

In some embodiments, a device is configured to determine the intensity of at least two specified wavelengths of light each at a substantially different (physical) location. In some embodiments, a device is configured to determine the intensity of at least two specified wavelengths of light each with a substantially different light-detection assembly. In some embodiments, a device is configured to determine the intensity of at least two specified wavelengths of light at substantially different locations of the same light-detection assembly.

In some embodiments, a light-detection assembly and associated detector window comprise at least one wavelength filter configured to pass only light having a specified wavelength of light. In some embodiments, a wavelength filter is functionally associated with a detector window. In some embodiments, a wavelength filter is a component of or is a detector window.

In some embodiments, a light-detection assembly and an associated detector window are configured so that light reaching the light-detection assembly is polarized, for example, the illuminator window is a light polarization component or the light-detection assembly comprises another light polarization component. In some such embodiments, the light polarization component associated with the light-detection assembly is oriented perpendicularly to the light polarization component associated with the illuminator. Such cross polarization reduces specular reflection and allows more selective acquisition of substantially only diffusely reflected light.

In some embodiments, a device comprises a single light-detection assembly.

In some embodiments, a device comprises at least two light detection assemblies. In some such embodiments, the device comprises at least one detector window associated with at least two of the detection assemblies. In some such embodiments, each light-detection assembly is associated with a single dedicated detector window

In some embodiments, at least one light-detection assembly is configured to determine the intensity of one specified wavelength of light. In some such embodiments, the light-detection assembly is functionally associated with a wavelength filter that limits the wavelengths of light that reach the light-detection assembly. In some embodiments, the wavelength filter is a component of or is the detector window.

In some embodiments, at least one light-detection assembly is configured to determine the intensity of at least two specified wavelengths of light.

In some such embodiments, the light-detection assembly configured to determine the intensity of at least two specified wavelengths of light is associated with at least two detector windows. In some such embodiments, each detector window is associated with a wavelength filter each passing light having a different specified wavelength.

In some such embodiments, the light-detection assembly configured to determine the intensity of at least two specified wavelengths of light is associated with a single detector window. In some such embodiments, the detector window is associated with the appropriate number of different wavelength filters, each passing light having a different specified wavelength.

The rate of determining the intensities of the specified wavelengths of light is any suitable rate. A higher rate produces more data (determined intensities) that must be analyzed and/or transmitted and/or stored but yields greater axial resolution by determining intensities at a greater rate as the device passes through the intestine. Considering the speed which peristalsis drives an ingested device through the gastrointestinal tract, it is currently believed that a rate of between 1 Hz and 20 Hz is preferred. Thus, in some embodiments, a light-detection assembly is configured to determine the intensity of the at least one specified wavelength of light at a rate of at least about 0.1 Hz, at least about 0.5 Hz and even at least about 1 Hz, preferably so that during the period of time that a device passes through the gastrointestinal tract substantially the entire luminal surface of a portion or the entire gastrointestinal tract downstream of the pylorus has been scanned.

Depending on the embodiments, a light-detection assembly can be configured to determine the intensity of at least one specified wavelength of light from any suitable direction and from any suitably-shaped region of a gastrointestinal wall.

In some embodiments, a light-detection assembly is configured to determine the intensity of light of at least one specified wavelength of light passing through an associated detector window from a circumferential section around the device axis of at least about 90°, at least about 120°, and even at least about 180° around the device axis, allowing simultaneous determination of intensities from an equivalent circumferential section (an arc-shaped region) of gastrointestinal luminal surface. That said, in preferred embodiments, a light-detection assembly is configured to determine the intensity of light in a circumferential section that is substantially the entire 360° around the device axis substantially simultaneously, allowing simultaneous determination of the intensity of reflection of substantially a 360° circumferential section (a ring-shaped region) of gastrointestinal tissue.

A detector window associated with a light-detection assembly allows light reflected from a given region of the gastrointestinal tract to the light-detection assembly. In some embodiments, the light-detection assembly is configured to determine the intensity of light from a single area that is substantially the entire region. For example, in some embodiments a device including a 360° circular detector window, light is reflected from a 360° ring-shaped region of the intestinal wall which is one area for which the intensity of reflected light is determined.

That said, in some embodiments, to provide additional information for diagnosis, for example, the location of an abnormality, the size of an abnormality or identification of multiple abnormalities at the same portion of the gastrointestinal tract, at least one light-detection assembly is configured to determine the intensity of at least one the specified wavelength of light passing through the associated detector window from at least two different areas, at least three, at least four, at least eight, at least ten, at least 15, at least 30, and even at least 60 different areas, for example, by an arrangement of a required number of light sensors in appropriate positions across from an associated detector window. For example, in some embodiments a device including a 360° circular detector window, a 360° ring-shaped region of the intestinal luminal wall is divided into two areas corresponding to two 180° sectors from which the intensity of light is independently determined, divided into three different areas corresponding to three 120° sectors, divided into four different area corresponding to four 90° sectors and the so on. In some such embodiments, the light-detection assembly comprises a pixelated light-detector array for determining the intensity of light from the different areas, the detector array comprising at least as many pixels as different areas. In some embodiments, a plurality of pixels are combined as a group to determine the intensity of light from one area. Any suitable technology of pixelated light-detector array may be used, e.g., monochrome pixelated arrays, CCD arrays, PDS arrays, PD arrays, CMOS arrays and LED arrays.

In this context, it is important to note that to acquire images having a diagnostically-useful resolution, known ingestible imaging devices generally acquire a frame made up of at least 10000 discrete areas (pixels), usually at least 1 million discrete areas (pixels). In contrast, a device as described herein is generally configured to simultaneously determine the intensity of not more than 1000, not more than 360 and even not more than 120 discrete areas at any one time. An advantage of such a seemingly low spatial resolution is that much less intense illumination light can be used (saving power), less data is acquired and still allowing detection of abnormalities.

In some embodiments, a light-detection assembly comprises a focusing component to concentrate light entering an associated detector window onto the pixelated light-detector array. In some embodiments, a focusing element is a component of or is the detector window.

In some embodiments, the apertures of the light detecting elements (pixels) of the light-detector array face an associated detector window. In some embodiments, a pixelated light-detector array has a circular periphery around which outwardly-facing light-detecting elements are arranged, in some such embodiments, so that the apertures of the light-detecting elements face the associated detector window.

In some embodiments, a light-detection assembly comprises a light-director to change the direction of light passing through an associated detector window towards a light-detector array. In some such embodiments, the light-detector array is substantially planar. Any suitable light-director may be used including a reflecting element (e.g., a mirror such as a substantially conical-section mirror), a light-guide, a prism, or a reflecting diffraction grating (e.g., a substantially conical-section diffraction grating).

In some embodiments, a light-director also functions as a wavelength separator (e.g., a prism (e.g., a conical section prism), a diffraction grating), in order to direct at least one specified wavelength of light towards a desired location of a light-detector array.

A detector window through which light passes to a light-detection assembly is substantially transparent to at least one of the specified wavelengths. As noted above, in some embodiments, a detector window is configured to act as a wavelength filter, for example is transparent to substantially only a single specified wavelength. As noted above, in some embodiments, a detector window is configured to act as a polarization light filter. A detector window is of any suitable shape and construction, and is generally fashioned of glass or plastic material as known in the art of ingestible imaging devices. In some embodiments, a detector window is made up of two or more discrete parts assembled so that the detector window is continuous. In some embodiments, a detector window is made up of two or more discrete parts assembled where at least two of the parts are separated by a non-transparent component. That said, it is generally preferred (for reasons of ease of construction as well as to reduce the chance of leakage of gastrointestinal fluids into the casing) that a detector window comprises substantially a single discrete component.

In some embodiments where a light-detection assembly is configured to determine the intensity of light from a certain circumferential section (e.g., 120°), the associated detector window comprises an arc or disk section of at least the same circumferential section of a material substantially transparent to at least one specified wavelength. In some embodiments, especially where an associated light-detection assembly is configured to determine the intensity of light from a substantially 360° circumferential section, the associated detector window is a ring or a disk of a material substantially transparent to at least one specified wavelength, in some embodiments positioned coaxial with the device axis.

In some embodiments, it is desired that one or more light-detection assemblies selectively detect substantially exclusively diffusely reflected light.

As noted above, in some such embodiments, the illuminator is functionally associated with a polarization component oriented in a first direction and a light-detection assembly is associated with a polarization component oriented perpendicularly to the first direction.

In some embodiments, the angular aperture of an illuminator in the plane of the device axis is relatively small so that little if any light specular reflected light is detected by a light-detection assembly. In some embodiments, the angular aperture of the illuminator in the plane of the device axis is less than about 30°, less than about 20°, less than about 10° and even less than about 5°. In some embodiments, the angular aperture of an illuminator in the plane of the device axis is limited by a lens (in some embodiments, the lens constituting the illuminator window) having the desired limited angular aperture. In some embodiments, the device comprises a narrow slit substantially perpendicular to the device axis in which the illuminator and/or the illuminator window are recessed and through which light must pass, thereby limiting the angular aperture of the illuminator in the plane of the device axis.

In some embodiments, the angular aperture of a light-detection assembly in the plane of the device axis is relatively small so that little if any light specular reflected light is detected by a light-detection assembly. In some embodiments, the angular aperture of the light-detection assembly in the plane of the device axis is less than about 30°, less than about 20°, less than about 10° and even less than about 5°. In some embodiments, the angular aperture of a light-detection assembly in the plane of the device axis is limited by a lens (in some embodiments, the lens constituting the associated detector window) having the desired limited angular aperture. In some embodiments, the device comprises a narrow slit substantially perpendicular to the device axis in which the light-detection assembly and/or the associated detection window are recessed and through which light must pass, thereby limiting the angular aperture of the light-detection assembly in the plane of the device axis.

In some embodiments, a light-detection assembly is functionally associated with a collimator so that light detected by the light-detection assembly first must pass the collimator, ensuring that little if any specular reflected light is detected by the light-detection assembly. In some embodiments, a collimator is a separate component. In some embodiments, a detector window is configured to also function as a collimator.

Design, construction, assembly and use of a device as described herein is apparent to a person having ordinary skill in the art upon perusal of the description and figures. Methods, materials and dimensions are similar to those used in the art of ingestible imaging devices such as the Pillcam™ (Given Imaging, Yokneam, Israel) and are easily modified to implement the teachings herein if necessary.

The dimensions of a device as described herein are any suitable dimensions, allowing passage through the gastrointestinal tract with causing excessive discomfort

That said, in some typical embodiments, a device has a total axial length of between about 15 mm and 35 mm, between about 20 mm and 30 mm, and even 25 mm like the Pillcam™.

In some typical embodiments, a body section of a device has an axial length of between about 10 mm and 30 mm, between about 10 mm and 20 mm, and even 15 mm like the Pillcam™.

In some typical embodiments, a body section is substantially cylindrical with a diameter of between about 5 mm and 20 mm, between about 7 mm and 15 mm, and even 10 mm like the Pillcam™.

The axial dimensions (axial length) and distance between the illuminator window and the detector window are any suitable values.

An illuminator window is of any suitable axial length, but typically not more than about 5 mm. Typically, an illuminator window is not less than about 0.3 mm long not less than about 0.5 mm long, and even not less than about 1 mm long.

A detector window is of any suitable axial length, but typically not more than about 4 mm long, not more than about 3 mm, not more than about 2 mm, and even not more than about 1 mm long.

In some embodiments, in order to collect sufficient light diffusely reflected from gastrointestinal tissue, the distance from an illuminator window to a detector window is as small as possible and in some embodiments, not more than about 5 mm, not more than about 4 mm, not more than about 3 mm, not more than about 2 mm and even not more than about 1 mm. Accordingly, in embodiments with multiple detector windows on the same side of an illumination window, the detector windows tend to have a small axial length (e.g., less than about 2 mm, less than about 1 mm) and to be close together, even substantially abutting.

As discussed above, the specified wavelength or wavelengths selected for implementing a device of method as discussed herein are selected to be diagnostic for some abnormality.

The spectral width of a specified wavelength is any suitable spectral width.

As understood from the discussion above (inter alia, FIGS. 1), in some embodiments a suitable spectral width is very broad, for example, in some embodiments a specified wavelength has a spectral width of:
up to about 400 nm (e.g., spans from about 400 nm to about 800 nm diagnostic for bleeding, FIG. 1A);
up to about 250 nm (e.g., spans from about 400 nm to about 650 nm diagnostic for bleeding, or from about 400 nm to about 625 nm diagnostic for invasive adenocarcinoma, FIG. 1A);
up to about 150 nm (e.g., spans from about 450 nm to about 600 nm diagnostic for adenomatous polyp or adenoma, FIG. 1B, for invasive adenocarcinoma, FIG. 1A, or hyperplastic polyp FIG. 1C or spans from about 500 nm to about 650 nm diagnostic for bleeding, FIG. 1A);
up to about 125 nm (e.g., spans from about 450 nm to 575 nm, diagnostic for invasive adenocarcinoma, or spans from about 500 nm to 625 nm, diagnostic for bleeding, FIG. 1A);
up to about 100 nm (e.g., spans from about 450 nm to 555 nm, diagnostic for invasive adenocarcinoma, or spans from about 500 nm to 600 nm, diagnostic for bleeding, FIG. 1A);
up to about 75 nm (e.g., spans from about 500 nm to 575 nm, diagnostic for bleeding, FIG. 1A); or
even up to about 50 nm (e.g., spans from about 525 nm to 575 nm, diagnostic for invasive adenocarcinoma, FIG. 1A or adenomatous polyp, FIG. 1B)

An advantage of such broad spectral widths is that the illuminator can be configured to produce a relatively low intensity of light, reducing energy use. A disadvantage of such broad spectral widths is the possibility that extraneous light (e.g., from external sources) will be detected as well as technical difficulty in implementing such broad spectral widths.

Thus, in some embodiments, a suitable spectral width is narrow, for example not more than about 10 nm FWHM, not more than about 5 nm FWHM and even not more than about 2 nm FWHM). Such narrow spectral widths are technically simple to implement using commercially-available wavelength filters.

A device as described herein may include any suitable illuminator. In some embodiments, it is preferred to use an illuminator of the invention. According to an aspect of some embodiments of the invention, there is provided an illuminator useful for projecting light in a radially-outwards direction in a 360° circumferential section comprising:
a) a light source for projecting light; and
b) a radial diffuser having a diffuser axis, a first face, a second face and a substantially circular circumferential outer edge coaxial with the central diffuser axis,
   wherein the light source is configured to project the light into the radial diffuser; and
   wherein at least a portion of the light projected into the radial diffuser from the light source radiates radially-outwards through the circumferential edge of the radial diffuser.

In some embodiments, the radial diffuser is disk-shaped, wherein at least a portion of the first face of the radial diffuser is substantially transparent to light projected by the light source; and wherein the light source is configured to project light into the radial diffuser through the transparent portion of the first face of the radial diffuser. In some such embodiments, the light source contacts the transparent portion of the first face of the radial diffuser.

In some embodiments, the radial diffuser is ring-shaped including a central hole with an inner rim; at least a portion of the inner rim is transparent to light projected by the light source; and wherein the light source is configured to project light into the radial diffuser through the inner rim. In some such embodiments, the light source contacts the transparent portion of the inner rim.

In some embodiments, the circumferential edge of the radial diffuser is perpendicular to the diffuser axis so that radially-outwards radiating light radiates substantially perpendicularly to the diffuser axis.

In some embodiments, the circumferential edge is oriented at an angle to the diffuser axis so that radially-outwards radiating light radiates at an angle relative to the diffuser axis.

In some embodiments, at least a portion of the first diffuser face is opaque to light projected by the light source. In some embodiments, substantially all of the first diffuser face is opaque to light projected by the light source. In some embodiments, at least a portion of the first diffuser face is light reflecting (e.g., mirrored).

In some embodiments, at least a portion of the second diffuser face is light reflecting (e.g., mirrored). In some embodiments, at least a portion of the second diffuser face is opaque to light projected by the light source. In some embodiments with a ring-shaped, substantially all of the second diffuser face is opaque to light projected by the light source.

As noted above, in some embodiments a device described herein comprises a light-detection assembly comprises a light-director to change the direction of light passing through an associated detector window towards a light-detector array and also to function as a wavelength separator. In some such embodiments, the light-director is a reflecting diffraction grating. In such embodiments, any suitable reflecting diffraction grating may be used. In some embodiments, it is preferred to use a diffraction grating of the invention. According to an aspect of some embodiments of the invention, there is provided a diffraction grating, comprising:

a substantially conical-section surface having an axis; and

on the surface, periodic features

wherein the surface and the periodic features are configured to reflect light impinging substantially perpendicularly to the axis at a wavelength-dependent angle in the general direction of the axis so that the diffraction grating functions as a dispersive element. In some embodiments, the periodic features comprise ring-shaped features coaxial to the axis. In some embodiments, the features comprise ring-shaped slits in the conical surface. In some embodiments, the features comprise ring-shaped ridges on the conical surface. In some embodiments, the surface has a substantially conical shape. In some embodiments, the diffraction grating has a substantially truncated conical shape.

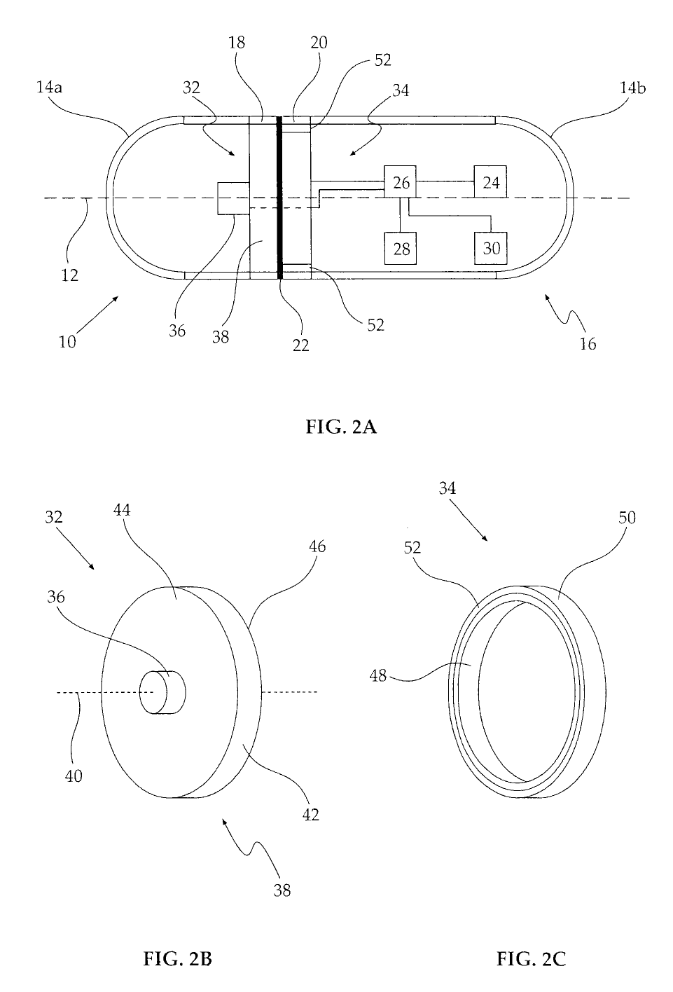

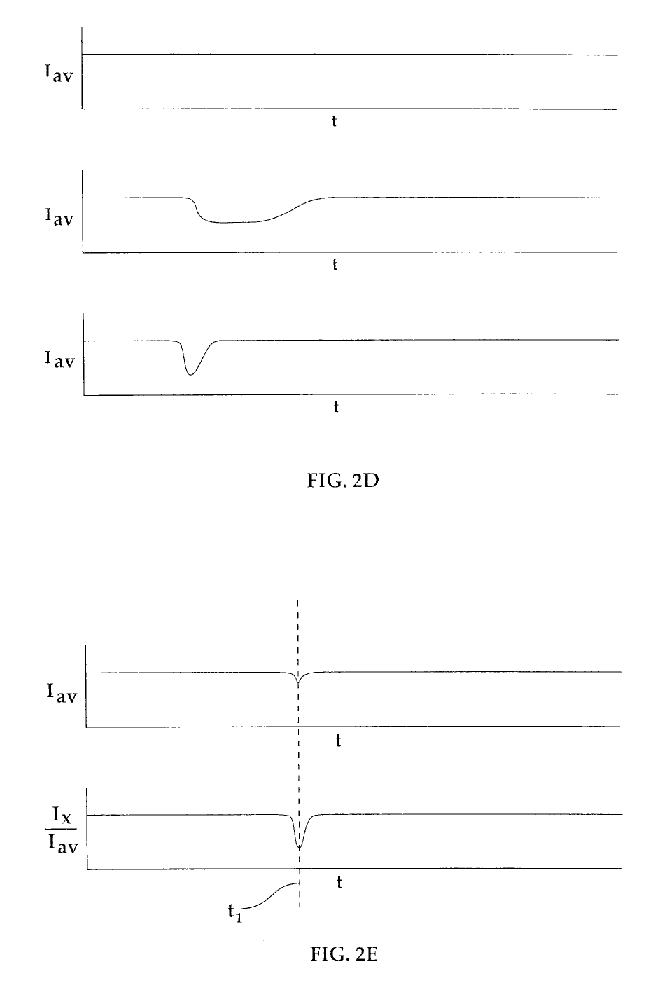

In FIGS. 2A-2C, an embodiment 10 of an ingestible device, is schematically depicted, in FIG. 2A device 10 in side cross-section, in FIG. 2B a detailed view of an illuminator of device 10 and in FIG. 2C a detailed view of a light-detection assembly of device 10. Device 10 is similar in dimensions and construction to a commercially available Pillcam™ (Given Imaging, Yoqneam, Israel).

The casing of device 10 includes a body axis 12, streamlined distal and proximal ends 14a and 14b and a parallel-walled cylindrical body section 16. The casing of device 10 is opaque to light except for illuminator window 18 and detector window 20, both 1 mm long complete rings of polycarbonate transparent to wavelengths of light between 400 nm and 800 nm, separated by separator 22, a 0.2 mm disk of opaque foil. Illuminator window 18 is configured to act as a polarizing component to polarize light in parallel to device axis 12. Detector window 20 is configured to act as a polarizing component to polarize light perpendicularly to device axis 12.

Inside body section 16 are a power supply 24 (e.g., a battery), a controller 26 (e.g., an integrated circuit also configured as a processor to process acquired data), a writeable memory 28 (e.g., a micro-SD card), a wireless transmitter 30 (e.g., a Bluetooth® transceiver), an illuminator 32 and a single light-detection assembly 34.

Illuminator 32 is configured to project light radially outwards through illuminator window 18 simultaneously in a 360° circumferential section around and perpendicular to axis 12. Illuminator 32, see FIG. 2B, comprises a light source 36 (e.g., in some embodiments an LED for producing white-light, in some embodiments producing polychromatic light, in some embodiments producing specified monochromatic light (e.g., 500 nm, 515 nm, 530 nm, 545 nm light) that receives electrical power for operation from power supply 24 through controller 26 and a radial diffuser 38, a disk of transparent material including a diffuser axis 40 and having a circular outer edge 42 parallel to diffuser axis 40. Radial diffuser 38 is configured to radially distribute light produced by light source 36 around diffuser axis 40 where the radially-outwardly radiating light radiates substantially perpendicularly to axis 40. A portion of a first face 44 of radial diffuser 38 where light source 36 contacts first face 44 is transparent to light produced by light source 36 so that light source 36 projects light into radial diffuser 38 through the transparent portion. The other portions of first face 44 as well as an entire surface of a second face 46 of radial diffuser 38 are completely mirrored (e.g., by deposition of a layer of silver or aluminum) and therefore opaque to light.

When activated, for example by controller 26, light source 36 produces light that enters radial diffuser 38 through the transparent portion of first face 44. The produced light is reflected inside radial diffuser 38 between the mirrored portions of first face 44 and second face 46 to emerge perpendicularly to diffuser axis 40 through outer edge 42 of radial diffuser 38 and through illuminator window 18, projecting a ring of light around device 10.

Light-detection assembly 34 of device 10 is associated with detector window 20 and is configured to determine the intensity of a single specified wavelength of light (e.g., 500 nm) passing through detector window 20 from twenty-four different areas of the luminal wall of the gastrointestinal tract each a 15° sector of a 360° circumferential section around axis 12, substantially simultaneously for all twenty-four areas. Light-detection assembly 34, see FIG. 2C, comprises a circular ring-shaped pixelated light-detector array 48 where many individual light-detectors are arranged on an outwards-facing periphery 50 of light-detector array 48 with apertures facing towards detector window 20. In some embodiments, light-detector array 48 is fashioned from a flexible photodetector array, see for example Yuan H-C et al in Appl. Phys. Lett. 94, 013102 (2009) or flexible photodetectors available from NANOIDENT Technologies AG, Linz, Austria.

Intimately contacting outwards-facing periphery 50 and covering the apertures of the light-detectors of light-detector array 48 is a ring of a narrow pass wavelength filter 52 chosen to selectively pass 500 nm light. Filter 52 is any suitable wavelength filter, for example a flexible filter available from Lee Filters, Andover, Hampshire, England.

Light-detection assembly 34 is functionally associated with controller 26, where the output of the individual light-detectors of light-detector array 48 are arranged in twenty-four groups, corresponding to the twenty-four different areas.

When light is produced by illuminator 32 is reflected from intestinal tissue towards detector window 20, only light having the specified wavelength of 500 nm passing through detector window 20 passes through wavelength filter 52 to impinge on the light-detectors of outwards-facing periphery 50 of light-detector array 48. At a rate of 10 Hz, controller 26 receives the intensity of 500 nm determined from each of the twenty-four groups of light-detectors of light-detector array 44.

For use, device 10 is activated and ingested by a subject, eventually passing the pylorus to enter and pass through the duodenum, small intestine, large intestine and rectum before being expelled through the anus. The location of the device in the gastrointestinal tract at any time is monitored in the usual way.

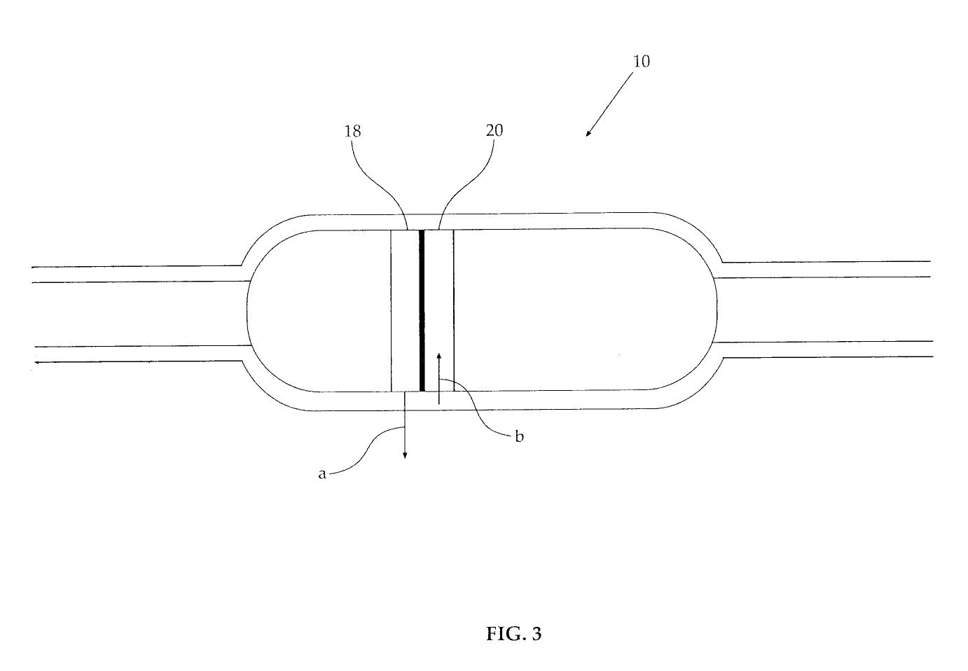

Illuminator 32 projects a ring of polarized light perpendicularly from illuminator window 18, illuminating a 360° circumferential section of gastrointestinal tissue in close proximity or touching illuminator window 18, arrow “a” in FIG. 3.

Some of the light is reflected from the gastrointestinal tissue towards detector window 20, arrow “b” in FIG. 3. Light reflected from a 360° circumferential section of gastrointestinal tissue of the specified wavelength (500 nm) passes through filter 52 to light-detector array 48. If present, most specular reflected light is prevented from passing through detector window 20 due to the cross polarization so that primarily diffusely projected light reaches light-detector array 48. The intensity of reflected light of the specified wavelength detected by each one of the twenty-four groups of light-detectors of light-detector array 44 (and thus of twenty-four different areas of gastrointestinal tissue, each constituting a 15° sectors of tissue) is determined at a rate of 10 Hz. Thus, the intensity of light of the specified wavelength passing through filter 52 that is reflected by the twenty-four areas of gastrointestinal tissue together constitute a complete ring of tissue encircling the device is detected by light-detector array 48 at a rate of 10 Hz and reported to controller 20.

The determined intensities are received by controller 26 and stored in an array in memory 28. When device 10 is expelled from the anus, the device is recovered and the recorded intensities constituting information indicative of a potential gastrointestinal abnormality can be downloaded for review and analysis to assist a medical professional in deciding whether there is an abnormality in the gastrointestinal tract of the subject.

Concurrently, controller 26 functions as a processor to compare the intensity of light reflected from the twenty-four different areas of gastrointestinal tract by normalizing each set of twenty-four simultaneously-determined intensities to the average of the twenty-four intensities. The normalized intensities constitute information indicative of a potential gastrointestinal abnormality are then transmitted to an appropriately-configured external unit (not depicted) outside of the body through wireless transmitter 30. An automatic program (e.g., written in Fortran programming language and running on a standard general purpose computer) analyzes the received normalized intensities to identify a relative intensity that is sufficiently low (e.g, less than about 60%, less than about 50% , less than about 30%) of the average, indicating a potential abnormality (e.g., bleeding, invasive adenocarcinoma). A medical professional can make a diagnosis based on information, including the normalized intensities or can choose to locate the area (or plurality of areas) for further investigation or treatment.

In some embodiments, the transmitted intensities are analyzed in real-time, and whenever a sufficiently low relative intensity is detected, the external unit sounds an audible alarm to warn an attending health-care professional.

In FIGS. 2D and 2E exemplary information acquired by a device such as device 10 is depicted.

In FIG. 2D, the average intensity of light having a wavelength of 500 nm reflected from gastrointestinal tissue and detected by all twenty-four groups of light-detectors is plotted as a function of time.

In the upper plot, the device passes through normal gastrointestinal tissue so the average determined intensity is substantially constant.

In the middle plot, the device passes through normal gastrointestinal tissue until encountering a bleeding lesion. At the bleeding lesion and downstream therefrom for a significant portion, the average determined intensity is substantially lower than of normal tissue. At some point in the intestine, the amount of blood on the intestinal surface becomes substantially lower and the average determined intensity returns to normal.

In the lower plot, the device passes through normal gastrointestinal tissue until encountering a localized invasive adenocarcinoma. The average determined intensity at the invasive adenocarcinoma is substantially lower than of normal tissue. When detector window 20 passes the invasive adenocarcinoma, the average determined intensity returns to normal.

The information such as depicted in FIG. 2D acquired in accordance with embodiments of the method and device described herein, when provided to a person such as a physician can be useful in helping making a diagnosis as to the presence and nature of gastrointestinal abnormalities. For example, together with other medical data, whether the subject is likely healthy, has intestinal bleeding or invasive adenocarcinoma.

In FIG. 2E, both plots are of information acquired by the same device 10 passing through the same portion of gastrointestinal tract. In the upper plot, the average determined intensity from all 24 groups of light-detectors is depicted. In the lower plot, the intensity of light determined from the light-detectors corresponding to a single group, corresponding to an area X of gastrointestinal tissue (a 15° sector), is depicted. From the upper plot, it is seen that at time t1 there is a slightly reduced average intensity, but study of the intensity of light determined reflected from area X show a significant reduced intensity at t1, indicative of invasive adenocarcinoma.

The information such as depicted in FIG. 2E acquired in accordance with embodiments of the method and device described herein, when provided to a person such as a physician can be useful in helping making a diagnosis as to the presence and nature of gastrointestinal abnormalities. For example, together with other medical data, whether the subject is likely healthy, has intestinal bleeding or invasive adenocarcinoma as well as the size of a potential abnormality (by the number of sectors that show a significant drop in intensity).

It is important to note that although device 10 is configured to determine the intensity of a very narrow selected wavelength (500 nm), other wavelengths can be used (as seen from FIGS. 1A-1C, as well as much broader selected wavelengths. For example, in some embodiments for detecting bleeding, illuminator 32 is configured to project monochromatic light with specified wavelength bandwidth, polychromatic light at specified wavelength range or white light and light-detection assembly 34 is configured to determine the intensity of a selected wavelength that spans from 400 nm to 800 nm without discrimination (useful for detecting bleeding). Analogously, some embodiments useful for detecting bleeding are configured to determine the intensity of a selected wavelength that spans from 500 nm to 650 nm, and any sub-group of selected wavelengths. Analogously, some embodiments useful for detecting invasive adenocarcinoma are configured to determine the intensity of a selected wavelength that spans from 400 nm to 600 nm, and any sub-group of selected wavelengths. Analogously, some embodiments useful for detecting adenomas or adenomatous polyps are configured to determine the intensity of a selected wavelength that spans from 475 nm to 575 nm, and any sub-group of selected wavelengths.

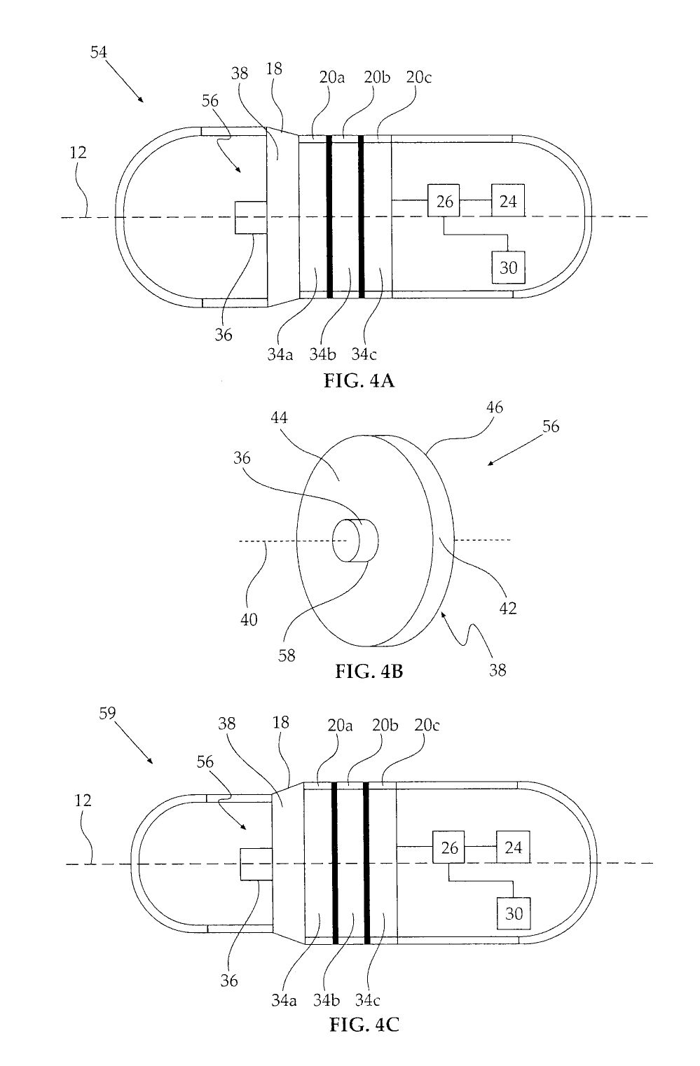

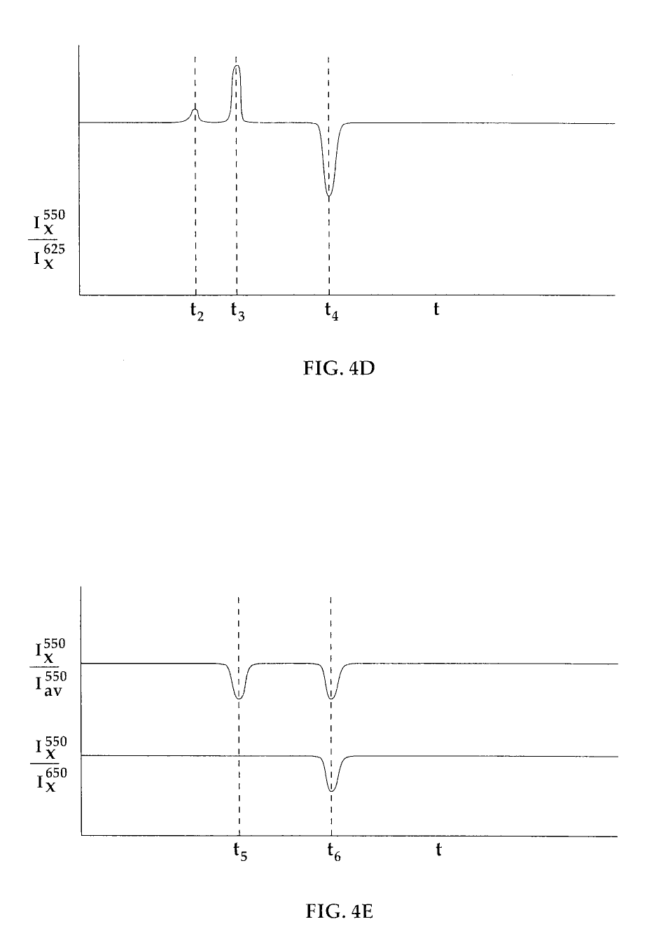

In FIGS. 4A-4B, an embodiment 54 of an ingestible device, is schematically depicted, in FIG. 4A, device 54 in side cross-section and in FIG. 4B a detailed view of a respective illuminator 56. Device 54 is similar to device 10 discussed above with a number of notable differences.

Whereas device 10 comprises a single light-detection assembly 34, device 54 comprises three independent light-detection assemblies 34a, 34b and 34c, each associated with a dedicated detector window 20a, 20b and 20c respectively. Light-detection assemblies 34 of device 54 are substantially identical to light-detection assembly 34 of device 10. However, instead of including a separate wavelength filter 52 as in device 10, each detector window 20 is fashioned of a colored polycarbonate material, thereby functioning as a wavelength filter. Consequently, each light-detection assembly 34 is configured to determine the intensity of a single specified wavelength of light, specifically, 500 nm, 550 nm and 650 nm. Consequently, device 54 is configured to determine the intensity of three specified wavelengths of light at substantially different physical locations, the respective light-detector arrays of the different light-detection assemblies 34a, 34b and 34c.

Like illuminator 32 of device 10, illuminator 56 of device 54 is configured to project light radially outwards through an illuminator window 18 simultaneously in a 360° circumferential section around axis 12. However, illuminator 56 is different in a number of significant ways from illuminator 32.

Illuminator 56, FIG. 4B, comprises a light source 36 (e.g., an LED for producing white-light or polychromatic light including 500 nm, 550 nm and 650 nm) and a radial diffuser 38, a ring including a central hole 58 of transparent material including a diffuser axis 40 and having a circular outer edge 42. The entirety of a first face 44 and second face 46 of radial diffuser 38 are mirrored and therefore opaque to light. The inner rim of central hole 58 of diffuser 38 is transparent to the light produced by light source 36, and light source 36 passes through central hole and contacts the inner rim, and is configured to project produced light into radial diffuser 38 through the inner rim.

Device 54 is devoid of a separate illuminator window component. Instead, radial diffuser 38 of illuminator 56 is configured to function as an illuminator window 18.

An additional significant difference is that outer edge 42 of radial diffuser 38 is not parallel to diffuser axis 40 but is oriented at an angle (5° from parallel) so that light radiating light radially-outwards from radial diffuser 38 radiates at an angle relative to diffuser axis 40 and body axis 12, in the general direction of light detection assemblies 34.

For use, device 54 is activated and ingested by a subject, eventually passing the pylorus to enter and pass through the duodenum, small intestine, large intestine and rectum before being expelled through the anus. The location of the device in the gastrointestinal tract at any time is monitored in the usual way.

Illuminator 32 projects a ring of white light at 5° from perpendicular from illuminator window 18, illuminating a 360° circumferential section of gastrointestinal tissue in close proximity or touching illuminator window 18.

Some of the light is diffusely reflected from gastrointestinal tissue towards detector windows 20a, 20b and 20c. Light reflected from a 360° circumferential section of gastrointestinal tissue of the three specified wavelengths (500 nm, 550 nm and 650 nm) passes through detector windows 20a, 20b and 20c and the intensities determined at a rate of 10 Hz by one of the twenty-four groups of light-detectors of the light-detector arrays 48 of the respective light detection assemblies 34a, 34b and 34c. Importantly, the light reflected from a given specific area of gastrointestinal tract is detected by a corresponding group of individual light-detectors of each light-detection assembly 34.

The determined intensities are received by controller 26. Controller 26 functions as a processor to compare the intensities of two different specified wavelengths reflected from the same area of the gastrointestinal tract. Specifically, for each cycle where intensities are determined and for each of the twenty-four groups of light-detectors corresponding to twenty-four discrete areas of gastrointestinal tract, controller 26 compares (e.g., by calculating a ratio) the intensities of 500 nm light, 550 nm light and of 650 nm light reflected from the same area. The comparisons of intensities constituting information indicative of a potential gastrointestinal abnormality are then transmitted to an appropriately-configured external unit (not depicted) outside of the body through wireless transmitter 30. An automatic program analyzes the received compared intensities (in some embodiments in real time, in some embodiments not in real time) to identify normalized intensities indicative of a potential abnormality. In some embodiments, device 54 transmits the determined intensities and the comparisons are made by the extremal unit.

By comparing the determined intensities of light at the different specified wavelengths 500 nm, 550 nm and 650 nm reflected from the same area of an intestinal wall, various gastrointestinal abnormalities can be potentially identified. For example, substantially similar intensities of light having wavelengths of 500 nm, 550 nm and/or 650 nm that is significantly lower than that of normal tissue is indicative of bleeding, while the pattern of a intensity of 650 nm greater than at 500 nm greater than at 550 nm is indicative of invasive adenocarcinoma, FIG. 1A. For example, when intensities of 500 nm and 550 nm higher than at 650 nm are indicative of adenomatous polyp while intensities of 500 nm and 550 nm lower than at 650 nm are indicative of invasive adeno carcinoma, FIG. 1B.

A medical professional receives the provided information and can then, optionally, make a diagnosis or act in a medically appropriate fashion.

In FIGS. 4D and 4E exemplary information acquired by a device such as device 54 is depicted.

In FIG. 4D, the ratio of the intensity of light having a wavelength of 550 nm (I_{X}⁵⁵⁰) to light having a wavelength of 650 nm)(I_{X}⁶⁵⁰) determined by corresponding groups of light-sensors of light-detection assemblies 34b and 34c reflected from the same area X of gastrointestinal tissue is plotted as a function of time.

At time t2, a slight increase of)(I_{X}⁵⁵⁰)/(I_{X}⁶⁵⁰) indicates the potential presence of a hyperplastic polyp (see FIG. 1B).

At time t3, a significant increase of (I_{X}⁵⁵⁰)/(I_{X}⁶⁵⁰) indicates the potential presence of a adenomatous polyp (see FIG. 1B).

At time t4, a significant decrease of))(I_{X}⁵⁵⁰)/(I_{X}⁶⁵⁰) indicates the potential presence of a invasive adenocarcinoma (see FIG. 1B).

In FIG. 4E, both plots are of information acquired by the same device 54 passing through the same portion of gastrointestinal tract.

In the upper plot of FIG. 4E, the ratio of intensity of 550 nm light determined by the group of light-sensors corresponding to area X to the average intensity of 550 nm light determined by all twenty-four groups of light-sensors. At times t5 and t6, a significant decrease in determined intensity indicates the potential presence of bleeding or invasive adenocarcinoma (see FIG. 1A).

In the lower plot of FIG. 4E, the ratio of the intensity of light having a wavelength of 550 nm to light having a wavelength of 650 nm determined by corresponding groups of light-sensors of light-detection assemblies 34b and 34c reflected from the same area X of gastrointestinal tissue is plotted as a function of time.

Taken together with information from the upper plot, at time t5 no substantial change in)(I_{X}⁵⁵⁰)/(I_{X}⁶⁵⁰) indicates the potential presence of bleeding (see FIG. 1A) while at time t6 a substantial decrease in (I_{X}⁵⁵⁰)/(I_{X}⁶⁵⁰)indicates the potential presence of invasive adenocarcinoma (see FIG. 1A).

The information such as depicted in FIGS. 4D and 4E acquired in accordance with embodiments of the method and device described herein, when provided to a person such as a physician can be useful in helping making a diagnosis as to the presence and nature of gastrointestinal abnormalities. For example, together with other medical data, whether the subject is likely healthy, has intestinal bleeding or has one of various stages of developing invasive adenocarcinoma.

In FIG. 4C, device 59 is schematically depicted. Device 59 is substantially identical to device 54 except that an illuminator 38 is configured to project light at a 5° in direction away from axis 12. Such projection reduces the amount of light that reaches detection-assemblies 34a, 34b and 34c, but reduces the intensity of specular reflection, and in some embodiments obviates some of the advantage of using polarizing components in illuminator 32 and light-detection assemblies 34.

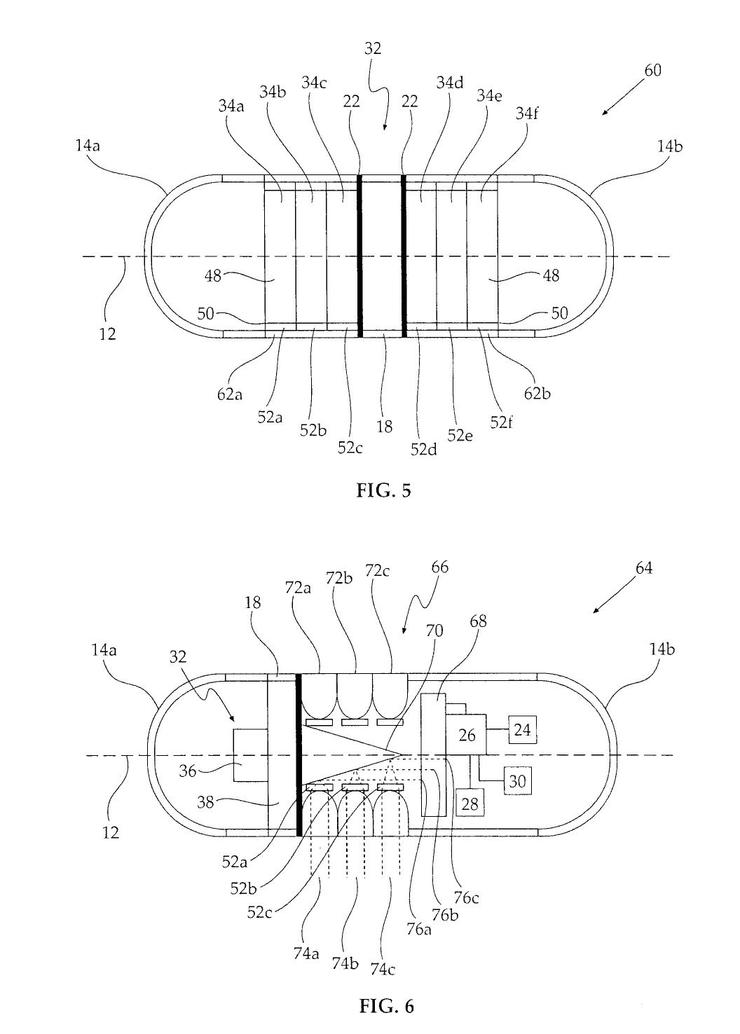

In FIG. 5, an embodiment 60 of an ingestible device, is schematically depicted in side cross-section.

Device 60 comprises an illuminator 32 substantially identical to illuminator 32 of device 10 configured to project light radially outwards through illuminator window 18 in a 360° circumferential section around and perpendicular to axis 12.

Device 60 also comprises six independent light-detection assemblies (34a, 34b, 34c, 34d, 34e and 34f) substantially identical to light-detection assembly 34 of device 10 depicted in FIG. 2C.

Device 60 comprises two substantially transparent detector windows 62a and 62b coaxial with device axis 12 on either side of illuminator window 18. Detector window 62a is associated with three light detection assemblies 34a, 34b and 34c while detector window 62b is associated with three light detection assemblies 34d, 34e and 34f.

As with devices 10 and 54, each light-detection assembly 20 of device 60 is configured to determine the intensity of one specified wavelength of light. Analogously to device 10, intimately contacting the outwards-facing periphery 50 of each light-detector array 48 of each light-detection assembly 34 is a ring of a narrow pass wavelength filter 52 chosen to pass only a specified wavelength of light. For example, in device 60, filters 52a and 52f pass the same specified wavelength of light (e.g., 500 nm), filters 52b and 52e pass the same specified wavelength of light (e.g., 550 nm) and filters 52c and 52d pass the same specified wavelength of light (e.g., 650 nm). In some related embodiments, a device is configured with six-light detection assemblies configured to determine the intensity of four, five or even six different specified wavelengths of light.

The use of device 60 is substantially as described above.

The embodiments of the ingestible devices described above comprise light detection assemblies, each configured to determine the intensity of a single specified wavelength of light. In some embodiments, a device comprises a light-detection assembly configured to determine the intensity of more than one, e.g., at least two specified wavelengths of lights.

In FIG. 6, an embodiment 64 of an ingestible device, is schematically depicted, in side cross-section.

Illuminator 32 of device 64 is substantially identical to illuminator 32 of device 10.

Device 64 comprises a single light-detection assembly 66 configured to determine the intensities of three different specified wavelengths of light.

Light-detection assembly 66 comprises three ring-shaped wavelength filters 52a (500 nm), 52b (550 nm) and 52c (650 nm) substantially similar to wavelength filter 52 of device 10, a planar light-detector array 68 (e.g., black-white CCD, PDA, CMOS, LED); for example a standard CCD array known in the art of digital photography) and a light-director 70, a reflecting element that is a conical-section mirror to change the direction of light passing through any of three detector windows 72a, 72b and 72c from substantially perpendicular to substantially in parallel to device axis 12 towards planar light-detector array 68.

Detector windows 72 are similar to detector window 20 of device 10, being rings of a material substantially transparent to the specified wavelengths of light. Additionally, detector windows 72 are configured as convergent lenses to focus light coming from any specific direction to a point on the surface of light director 70, each having a different focal length so that each window 72 focuses light at the surface of light director 70 across from a respective window 72. Windows 72 are configured to have an angular aperture in the plane of axis 12 of ±3° from perpendicular to axis 12.

For use, device 64 is activated and ingested by a subject, eventually passing the pylorus to enter and pass through the duodenum, small intestine, large intestine and rectum before being expelled through the anus. The location of the device in the gastrointestinal tract at any time is monitored in the usual way.

Illuminator 32 projects a ring of white light perpendicularly from illuminator window 18, illuminating a 360° circumferential section of gastrointestinal tissue in close proximity or touching illuminator window 18.

Some of the light is (diffusely) reflected from the gastrointestinal tissue towards detector windows 72a, 72b and 72c. As indicated by traces 74a, 74b and 74c, light reflected from a 360° circumferential section of gastrointestinal tissue around each window 72, passes through the window and is focused through a respective wavelength filter 52 towards the surface of light director 70. Consequently, each of the three specified wavelengths of light is focused onto a separate ring-shaped section of the reflecting surface of light director 70, to be reflected at about 90° towards light-detector array 68. As a result, three concentric circles 76a (500 nm), 76b (550 nm) and 76c (650 nm) of light are reflected onto the surface of light-detector array 68, each circle corresponding to a different one of the three specified wavelengths of light. Device 64 is thus configured to determine the intensity of the three specified wavelengths of light, each at a substantially different location of light-detector array 68.

The intensities are determined at a rate of 10 Hz where the directional resolution (the number of areas into which the 360° circumferential section of gastrointestinal tissue is divided) of each specified wavelength of light can be selected as desired, limited only by the pixel resolution of light-detector array 68 and the radius of a concentric circle 76 corresponding to the specified wavelength of light.

The determined intensities are received by controller 26 and are further handled as required, for example, substantially as described with relation to device 54.

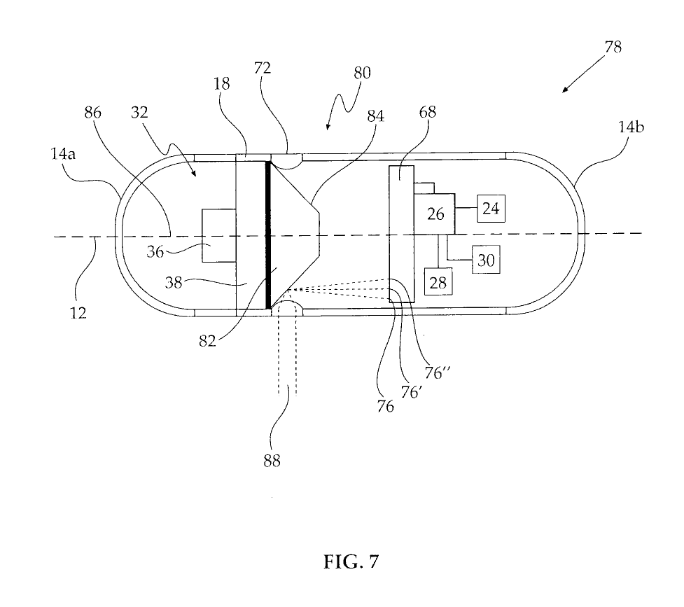

In FIG. 7, an embodiment 78 of an ingestible device comprising a single light-detection assembly configured to detect multiple specified wavelengths of light, is schematically depicted, in side cross-section.

Illuminator 32 of device 78 is substantially identical to illuminator 32 of device 10.

Device 78 comprises a single light-detection assembly 80 configured to determine the intensities of multiple specified wavelengths of light.

Light-detection assembly 80 comprises a planar light-detector array 68 substantially similar to light-detector array 68 of device 64 and a light-director 82, a reflecting element that is a conical-section diffraction grating to change the direction of light passing through the single detector window 72 of device 78 from substantially perpendicular to device axis 12 towards planar light-detector array 68 at a wavelength-dependent angle.

Specifically, light-director 82 is a conical-section diffraction grating with an outer surface 84 having the shape of a truncated cone, see FIG. 7) on which are periodic features comprising ring-shaped slits and ridges coaxial to axis 86 of light-director 82. The periodic features have dimensions and are spaced in the manner of diffraction gratings so that surface 84 functions as a wavelength-dependent dispersive optical element.

Detector window 72 is substantially identical to detector windows 72 of device 64.

For use, device 80 is activated and ingested by a subject, eventually passing the pylorus to enter and pass through the duodenum, small intestine, large intestine and rectum before being expelled through the anus. The location of the device in the gastrointestinal tract at any time is monitored in the usual way.

Illuminator 32 projects a ring of white light perpendicularly from illuminator window 18, illuminating a 360° circumferential section of gastrointestinal tissue in close proximity or touching illuminator window 18.

Some of the light is reflected from the gastrointestinal tissue towards detector window 72. As indicated by trace 88, light reflected from a 360° circumferential section of gastrointestinal tissue around window 72, passes through window 72 and is focused towards the surface of light director 82. Consequently, all light passing through window 72 is focused into a ring-shaped section of surface 84 of light director 82. Functioning as a dispersive optical element, light director 82 reflects the light impinging substantially perpendicularly to axis 86 at a wavelength-dependent angle in the general direction of axis 86 towards light-detector array 68. As a result, a continuum of concentric circles 76 of light are reflected onto the surface of light-detector array 68, each circle corresponding to a different wavelength of light. Device 78 is thus configured to determine the intensity of a plurality of specified wavelengths of light, each at a substantially different physical location of light-detector array 68. In such a way, device 78 is configured to separate light entering detector window 72 into constituent wavelength components and thus acquires a spectrum of each area of gastrointestinal tissue, where the exact number of specified wavelengths and the spectral resolution can be selected as desired, limited only by the pixel resolution of light-detector array 68 and the radius of a concentric circle 76 corresponding to the specified wavelength of light.

The intensities are determined at a rate of 10 Hz, where the directional resolution (the number of areas into which the 360° circumferential section of gastrointestinal tissue is divided) of each specified wavelength of light can be selected as desired, limited only by the pixel resolution of light-detector array 68 and the radius of a concentric circle 76 corresponding to the specified wavelength of light.

The determined intensities are received by controller 26 and are further handled as required, for example, substantially as described with relation to device 54.

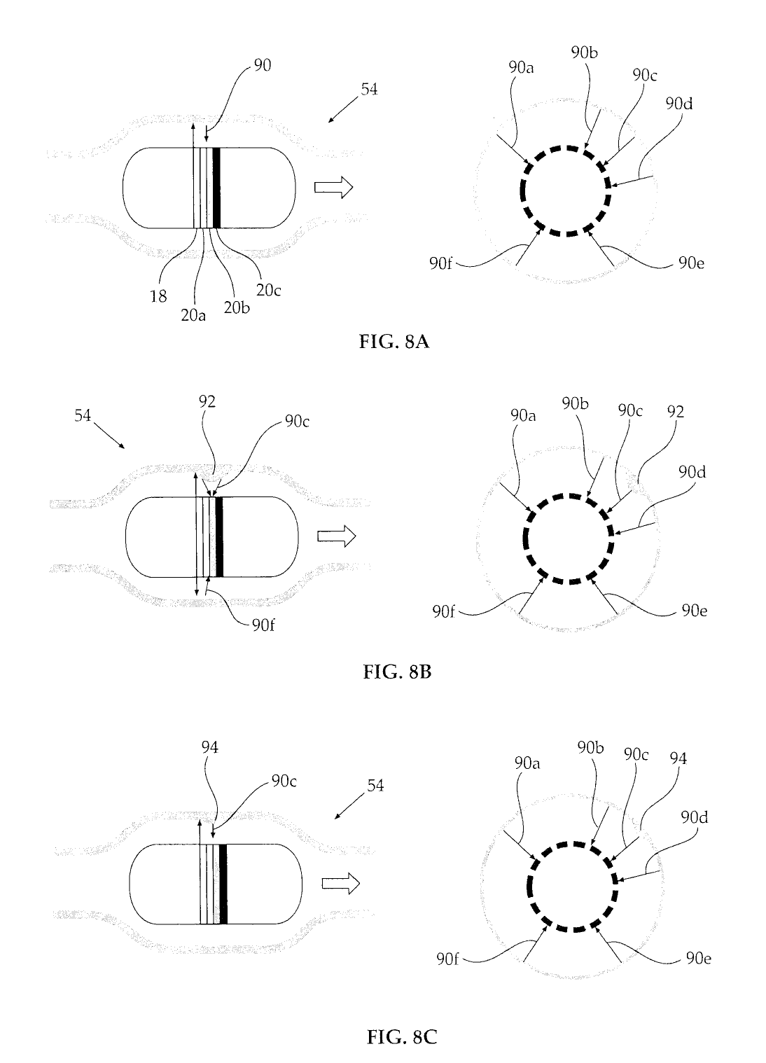

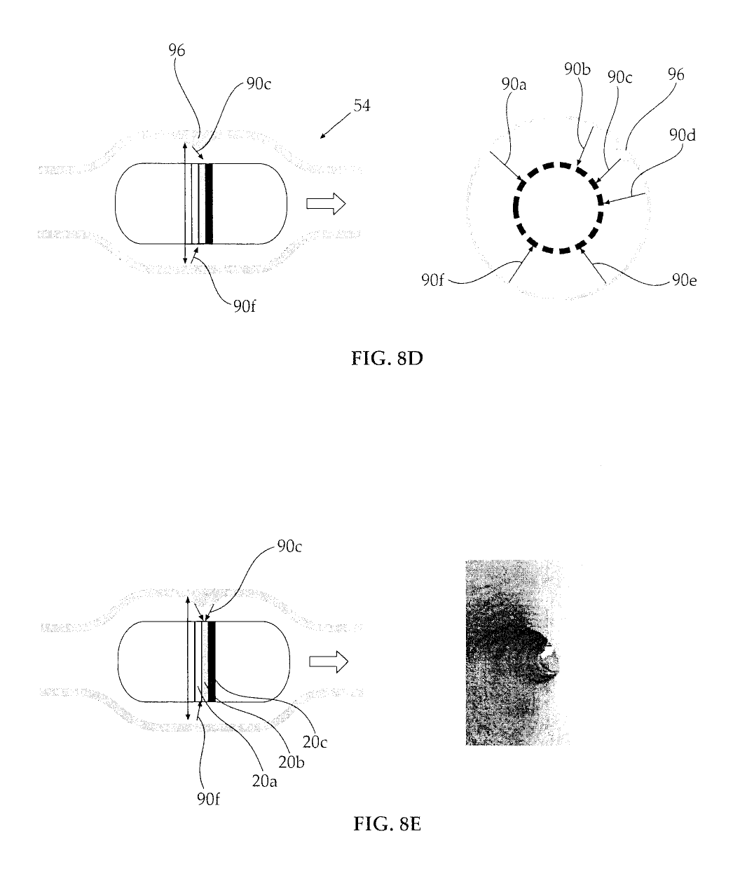

In FIGS. 8A-8E, the use of an embodiment of a device as described herein in implementing embodiments of the method described herein are discussed. Specifically, changes in detected intensities of reflection are correlated with the distance of an intestinal wall to a light-detection assembly, allowing identification of protruding surface features and the like. In FIGS. 8, a device 54 configured for the detection of blood including three independent light-detection assemblies 34 each configured with an associated detector window 20a, 20b and 20c to determine the intensity of a single specified wavelength of light is depicted inside a gastrointestinal tract while being used for implementing an embodiment of the method described herein. Specifically, light-detection assemblies 34a associated with detector window 20a, 34b associated with detector window 20b and 34c associated with detector window 20c are configured to detect wavelengths of 500 nm, 710 nm and 740 nm, respectively. Generally, light projected in a direction through illuminator window 18 is reflected from gastrointestinal tissue to be detected by each light-detection assembly 34. The intensity of a given wavelength detected by a given light-detection assembly 34 from a given direction 90 is dependent on both the distance from the reflecting surface and the spectral characteristics of the tissue.

In FIG. 8B, all areas of tissue have normal spectral characteristics but a protruding abnormality 92 (e.g., a benign polyp) reflects light differently from normal tissue. Specifically, as device 54 passes along abnormality 92, the individual detectors in the sector (e.g., direction 90c ) across abnormality 92 of all three light-detection assemblies 34 register a similar difference in detected intensity relative to other sectors (e.g., 90a, 90b, 90d, 90e, 90f)

In FIG. 8C, all areas of tissue are at a substantially similar distance, but a substantially non-protruding abnormality 94 (e.g., bleeding) has spectral characteristics different from those of normal tissue, therefore reflecting light differently from normal tissue. Specifically, as device 54 passes along abnormality 94, the individual detectors in the sector (e.g., direction 90c) across abnormality 94 of the three light-detection assemblies 34 register a reduction in intensity, each specified wavelength to a different degree (FIG. 1A).

In FIG. 8D, a protruding abnormality 96 having spectral characteristics different from those of normal tissue (e.g., a bleeding polyp) reflects light differently from normal tissue. Specifically, as device 54 passes along abnormality 96, the individual detectors in the sector (e.g., direction 90c ) across abnormality 96 of the three light-detection assemblies 34 register a reduction in intensity, each specified wavelength to a different degree (FIG. 1A) but also different from a non-protruding abnormality such as 94.

In FIG. 8E, is seen that changes in intensity caused by differences in distance to detector windows 20 can subsequently be assembled to create a virtual image, for example using pipe simulation mathematics.

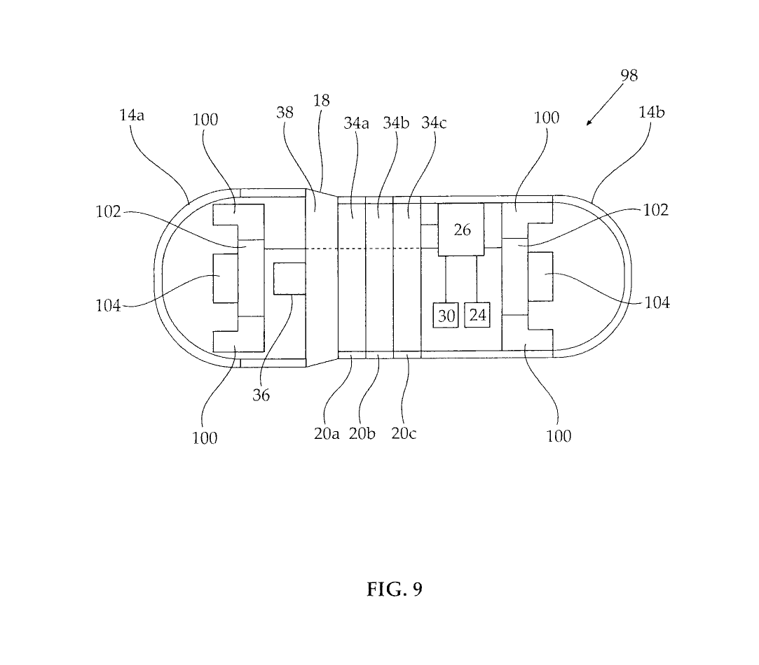

In some embodiments, a casing of a device as described herein further comprises the components required to add the functionality of an ingestible gastrointestinal imaging device, e.g., a Pillcam™ (Given Imaging, Yokneam, Israel) so that casing contains both a device as described herein and an ingestible gastrointestinal imaging device, yielding a combined device including the abnormality-detection functionality described herein together with an imaging functionality. An embodiment of such a combined device, device 98, is schematically depicted in FIG. 9. Device 98 includes all the required components of device 54 depicted in FIGS. 4, as well as imaging components including transparent proximal and distal ends 14a and 14b, image acquisition light sources 100, and cameras 102 with objective lenses 104.

In some such embodiments, detection of a suspected abnormality in accordance with the teachings herein optionally triggers the imaging functionality.

For example, in some such embodiments, the imaging functionality is only activated as a result of detection of a suspected abnormality. After the device is ingested, only the abnormality-detection functionality is active. When a suspected abnormality is detected, the imaging functionality, typically of an upstream (backwards) facing camera, is activated. Such embodiments use the power required for operating the camera and camera light source only when needed, reducing the number of images acquired that must be transmitted and/or and stored, saving power and therefore allowing reduction of the size of the power source as well as saving time and expense of the professional who must review the images.

For example, in some such embodiments, the quality of images acquired by an imaging functionality is changed as a result of detection of a suspected abnormality. After the device is ingested, both the imaging and the abnormality detection functionality are active. When a suspected abnormality is detected, the quality of images acquired is increased, for example by increasing resolution, frame rate or illumination intensity, typically of an upstream (backwards) facing camera.

For example, in some such embodiments, the results of abnormality detection are used to identify which images should be reviewed with greater attention. As is well-known, the images acquired by known ingestible devices must be reviewed by a health-care professional in order to identify abnormalities. As the images are acquired as a long and uninteresting film, the health-care professional may not see an abnormality. After the device is ingested, both the imaging and the abnormality detection functionality are active. Images acquired in proximity to where an abnormality is detected are noted (e.g., in a separate list, one or more the images is marked). A person reviewing the images is able to choose to review the noted images with extra care.

### EXAMPLES

### Example 1

### Wavelength Dependence of Diffuse Reflection from Normal and Abnormal Tissue

The wavelength dependence of diffuse reflection from the intestinal abnormalities of blood and invasive adenocarcinoma relative to normal intestinal mucosa was examined in a manner simulating the use of an ingestible device as described herein.

Two freshly excised samples (about 20 cm by 40 cm) of human intestinal tissue were provided, one with a bleeding area and one having an invasive adenocarcinoma, as determined by a pathologist.

As an illuminator, a 0.6 mm) diameter glass fiber was connected to an SE NET Model I-150 fiber optic light source including a 150 Watt Quartz halogen lamp and the distal tip of the fiber contacted with an area of intestinal tissue. As a light-detection assembly, a 0.2 mm diameter glass fiber was connected to a StellarNet Green Fiberoptic spectrometer (StellarNet, Inc, Tampa, Fla., USA) and the distal tip contacted with the intestinal tissue, 0.2 mm from the illuminator glass fiber. As the distal tip of the light detection glass fiber contacted the tissue, only diffusely reflected light was guided into and detected by the spectrometer.

The distal tips of the glass fibers were contacted with a bleeding area of the first sample of intestinal tissue and the spectrometer activated to detect the intensity of light diffusely reflected from the bleeding area from 400 nm to 800 nm at increments of 1 nm. The intensity measurements were repeated with an area of intact mucosa of the same intestinal tissue sample. The relative detected intensities are depicted in FIG. 1A, normalized relative to detected intensities of the intact mucosa, of the intact mucosa (plot ‘a’) and of the bleeding area (plot ‘b’).

The distal tips of the glass fibers were contacted with an area of the second sample of intestinal tissue having the invasive adenocarcinoma and the spectrometer activated to detect the intensity of light diffusely reflected from the invasive adenocarcinoma from 400 nm to 800 nm at increments of 1 nm. The intensity measurements were repeated with an area of intact mucosa of the same intestinal tissue sample. The relative detected intensities are depicted in FIG. 1A, normalized relative to detected intensities of the intact mucosa, of the invasive adenocarcinoma (plot ‘c’).

It is apparent from FIG. 1A, that the diffuse reflection of different tissue types has distinct spectral characteristics, and that the teachings herein may be used to provide information useful for assisting in diagnosis of gastrointestinal abnormalities.

### Example 2

### Wavelength Dependence of Diffuse Reflection from Normal and Abnormal Tissue

The wavelength dependence of diffuse reflection from various intestinal abnormalities related to invasive adenocarcinoma relative to normal intestinal mucosa were examined in a manner simulating the use of an ingestible device as described herein.

Fifty-four in vivo spectral measurements were performed during standard colonoscopy in patients having various abnormalities as determined by a physician (endoscopist) and confirmed by a pathologist.

As an illuminator, a standard light source (xenon arc lamp) of endoscopic system (Olympus CV180, Olympus, Japan) was used. Light was directed at intestinal tissue to illuminate the entire surface of the tissue. A light-detection assembly includes a 600μ (0.6 mm) diameter glass fiber connected to a StellarNet Green Fiberoptic spectrometer (StellarNet, Inc, Tampa, Fla., USA) as described in Example 1. As the distal tip of the light detection glass fiber contacted the tissue, only diffusely reflected light was guided into and detected by the spectrometer.

For each area of tissue, the distal tip of the light-detection glass fiber was first contacted with an area of an identified intestinal abnormality and the intensity of diffusely reflected light determined between 400 nm and 750 nm at 1 nm increments. The intensity measurements were repeated with an area of intact mucosa of the same intestinal tissue sample. The determined intensities of the abnormal tissue were normalized relative to the determined intensities of the normal tissue.

The relative detected intensities are depicted in FIG. 1B, of intact mucosa (plot ‘a’), invasive adenocarcinoma (plot ‘c’, average of 5 samples), hyperplastic polyp (plot ‘d’, average of 6 samples), adenomatous polyp (plot ‘e’, average of 20 samples) and adenoma (plot ‘f’, average of 23 samples).

It is apparent from FIG. 1B, that the diffuse reflection of different tissue types has distinct spectral characteristics, and that the teachings herein may be used to provide information useful for assisting in diagnosis of gastrointestinal abnormalities.

### Example 3

### Wavelength Dependence of Diffuse Reflection from Normal and Abnormal Tissue

The wavelength dependence of diffuse reflection from various intestinal abnormalities relative to normal intestinal mucosa was examined in a manner simulating the use of an ingestible device as described herein.

45 freshly excised samples (about 20 cm by 40 cm) of human intestinal tissue were provided, having various abnormalities as determined by a pathologist.

As an illuminator, a quartz halogen lamp with polarizing filter were directed at a sample of intestinal tissue to illuminate the entire surface of the tissue. A spectral camera (SD-300 from Applied Spectral Imaging, Migdal Haemek, Israel) with a polarizing filter oriented perpendicularly to the polarizing filter of the illuminator was used as a light-detection assembly with the objective lens positioned 10 cm from a tissue sample surface to acquire spectra using the polarized-gated method between 400 nm and 800 nm at increments of 1 nm of areas of the surface of each of the samples. As the illuminator and light-detection assembly were cross-polarized, the light detected was primarily diffusely reflected light.

The spectra of areas of abnormal tissue corresponding to bleeding tissue (11 samples), invasive adenocarcinoma (5 samples), hyperplastic polyps (6 samples) and adenoma (23 samples) were normalized relative to the spectra of nearby normal tissue from the same sample.

The spectra of each of the abnormal tissue types and the normal tissue were averaged and displayed in FIG. 1C: normal tissue (plot ‘a’, average of 45 spectra), bleeding (plot ‘b’, average of 11 spectra) invasive adenocarcinoma (plot ‘c’, average of 5 spectra), hyperplastic polyps (plot ‘d’, average of 6 spectra) and adenoma (plot ‘f’, average of 23 spectra).

It is apparent from FIG. 1C, that the diffuse reflection of different tissue types has distinct spectral characteristics, and that the teachings herein may be used to provide information useful for assisting in diagnosis of gastrointestinal abnormalities.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

For example, in the embodiments described above, the devices comprise an illuminator with a radial diffuser giving a homogenous illumination around the device. In some embodiments, other types of illuminators are used, for example, illuminating strips or a circular array of outwardly-facing light sources such as LEDs.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Citation or identification of any reference in this application shall not be construed as an admission that such reference is available as prior art to the invention.

Section headings are used herein to ease understanding of the specification and should not be construed as necessarily limiting.

1. An ingestible device useful for providing information useful for diagnosis of gastrointestinal abnormalities, comprising:
a) an ingestible casing having a device axis, a body section, a distal end and a proximal end;
b) inside said casing, an illuminator configured to project light radially outwards through an illuminator window of said housing; and
c) inside said casing, at least one light-detection assembly configured to determine intensity of at least one specified wavelength of light projected by said illuminator and passing through an associated detector window after reflection from a portion of a gastrointestinal tract without acquiring an image.

2. The device of claim 1, wherein said illuminator and said illuminator window are configured together to project light substantially perpendicularly to said device axis.

3. The device of claim 1, wherein said illuminator and said illuminator window are configured together to project light at an angle less than 90° to said device axis.

4. The device of claim 1, wherein said illuminator is configured to project light in circumferential section around said device axis of at least about 90°.

5. The device of claim 4, wherein said circumferential section is substantially 360° around said device axis.

6. The device of claim 5, wherein said illuminator window is of a material substantially transparent to said at least one specified wavelengths of light, having a shape selected from the group of a ring and a disk.

7. The device of 1, further comprising a light source for producing light including light having said at least one specified wavelengths.

8. The device of claim 7, further comprising a radial diffuser functionally associated with said light source to radially distribute light produced by said light source.

9. The device of claim 1, wherein said at least one light-detection assembly and said associated detector window comprising at least one wavelength filter configured to pass only light having said at least one specified wavelength of light.

10. The device of claim 1, comprising a single said light-detection assembly

11. The device of claim 1, comprising at least two said light-detection assemblies

12. The device of claim 1, wherein said at least one light-detection assembly is configured to determine the intensity of one specified wavelength of light

13. The device of claim 1, wherein said at least one light-detection assembly is configured to determine the intensity of at least two specified wavelengths of light

14. The device of claim 1, wherein said at least one light-detection assembly is configured to determine the intensity of said at least one specified wavelength of light passing through said associated detector window from a circumferential section around said device axis of at least about 90°.

15. The device of claim 14, wherein said circumferential section is substantially 360° around said device axis.

16. The device of claim 1, wherein said at least one light-detection assembly is configured to determine the intensity of said at least one specified wavelength of light passing through said associated detector window from at least four different directions.

17. The device of claim 16, further comprising a pixelated light-detector array for determining said intensity of light from said different directions.

18. The device of claim 17, wherein pixels of said light-detector array face said associated detector window.

19. The device of claims 17, wherein said light-detector array has a circular periphery around which outwardly facing pixels are arrayed

20. The device of claim 1, wherein said device is configured to determine the intensity of at least two specified wavelengths of light each at a substantially different location.

21. The device of claim 1, wherein said device is configured to determine the intensity of at least two specified wavelengths of light each with a substantially different light-detection assembly.

22. The device of claim 1, wherein said light from said illuminator is projected substantially simultaneously in a substantially 360° circumferential section around said device axis;

23. An illuminator useful for projecting light in a radially-outwards direction in a 360° circumferential section comprising:
a) a light source for projecting light; and
b) a radial diffuser having a central diffuser axis, a first face, a second face and a
substantially circular circumferential outer edge coaxial with said central diffuser axis,
wherein said light source is configured to project said light into said radial diffuser; and
wherein at least a portion of said light projected into said radial diffuser from said light source radiates radially-outwards through said circumferential edge.

24. A diffraction grating, comprising:
a substantially conical-section surface having an axis; and
on said surface, periodic features
wherein said surface and said features are configured to reflect light impinging on said surface substantially perpendicularly to said axis at a wavelength-dependent angle in a general direction of said axis so that the diffraction grating functions as an optically dispersive element.

25. A method for providing information useful for diagnosis of gastrointestinal abnormalities, comprising:
a) illuminating an area of an in vivo gastrointestinal tract of a living mammal with light, wherein said illuminating is outwards from inside the a lumen of the gastrointestinal tract;
b) without acquiring an image of said area, determining an intensity of at least one specified wavelength of light after said light is reflected from said area of said gastrointestinal tract; and
c) providing information related to said intensity of light indicative of a potential gastrointestinal abnormality in said area.

26. The method of claim 25, wherein said reflected light is substantially diffusely reflected light.

27. The method of claim 25, wherein said illumination is substantially simultaneous for a plurality of discrete areas.

28. The method of claim 27, wherein said plurality of discrete areas constitute a circumferential section of said gastrointestinal tract.

29. The method of claim 27, wherein said plurality of discrete areas constitute a 360° circumferential section of said gastrointestinal tract.

30. The method of claim 25, wherein said information provided is a comparison of at least two respective said intensities of at least two different specified wavelengths reflected from the same said area.

31. The method of claim 25, wherein said information provided is said intensity compared to an intensity of light of a same specified wavelength after said light has been reflected from a different area of said gastrointestinal tract.
